SEQUENCE LISTING (6 sequences)

SEQ ID NO: 1 - DNA, 20 bp, Artificial
  synthetic forward primer 1 for  Mus musculus CYP1A1 gene
cagagagcacctgcaaaaca

SEQ ID NO: 2 - DNA, 20 bp, Artificial
  synthetic reverse primer 1 for  Mus musculus CYP1A1 gene
ggctacaaagggtgatgctt

SEQ ID NO: 3 - DNA, 27 bp, Artificial
  synthetic forward primer 2 for  Mus musculus CYP1A1 gene
ctcgaggagggcaggtgaaggtgttag

SEQ ID NO: 4 - DNA, 27 bp, Artificial
  synthetic reverse primer 2 for  Mus musculus CYP1A1 gene
aagcttaagtgaagagtgttctctagg

SEQ ID NO: 5 - DNA, 479 bp, Artificial
  amplified PCR product of forward primer 1 and reverse primer 1
cagagagcacctgcaaaacagccagctaggcgtgccgggtggtgctgcgtgtcctgccactaaaaatgtggacacacgctgttcaggcctttgctctcaggcagaagccgagcatcgcacgcaaacccggccctttgcaccgctggcgctgtcttgtcgcgcccttgcaaagcatagactatttctatctcttaaaccccaccccaacgccccaggagagctggccctttaagagcctcacccacggttcccctcccccagctagcgtgacagcactgggacccgcgcccggttgtgagttgggtagctgggtggctgcgcgggcctccaggctcttctcacgcaactccggggcaccttgtccccagccaggtggggcggagacaggcagcccgacctctgcccccagaggatggagcaggcttacgcacgctagcctcaggaacctgtgtgcgtgccaagcatcaccctttgtagcc

SEQ ID NO: 6 - DNA, 1503 bp, Artificial
  amplified PCR product of forward primer 2 and reverse primer 2
ctcgaggagggcaggtgaaggtgttagttaggaacaggttgagttagacacgccaagttcagatgttcttcttacactaatctcactctgggagcacccttcagggccagagagcacctgcaaaacagccagctaggcgtgccgggtggtgctgcgtgtcctgccactaaaaatgtggacacacgctgttcaggcctttgctctcaggcagaagccgagcatcgcacgcaaacccggccctttgcaccgctggcgctgtcttgtcgcgcccttgcaaagcatagactatttctatctcttaaaccccaccccaacgccccaggagagctggccctttaagagcctcacccacggttcccctcccccagctagcgtgacagcactgggacccgcgcccggttgtgagttgggtagctgggtggctgcgcgggcctccaggctcttctcacgcaactccggggcaccttgtccccagccaggtggggcggagacaggcagcccgacctctgcccccagaggatggagcaggcttacgcacgctagcctcaggaacctgtgtgcgtgccaagcatcaccctttgtagccccagaccccctcctgctgtctcgcgtggatccttcctccaccctttcctccaccatacttagatagctctgcacccgccgcccgacttctctgtgttgcctgcttcagtatgtatgcacaacactagcaagccccgggagcctacagggagctgcaagcggggactcccgggcttgcagcttgcctaaggtgacccttagaggtgagatctgcaagcctgccatccatccccaccctctagatgaagcagcgcgaacttcggccgatacccaatttgtggggcacagagtcagtccaatggcgccactggccttcctgtcctgtgacctctgggctggggtcgttgcgcttctcacgcgagcttggactcagtaatccagggaagcaaagtcaccacccagctgttctccctctaccagcctttcccgggcacccattggcttgtagtaggcaagaggatcttacacactgaatgtttcaggggtgcaaaaatagtgaaaaggaatccctatgacccggaatggaggccccagtacttactttttaggttaccccagaatttttcctcaaacccctccctcagtgggattatgcactgtccatggagcaccttgaaagtgtggggggtggtgaccccaacctttattctttttcttttcttatgtttttttgtgcctgtacctacatcaggtatccggtatggcttcttgcctatctcccccctcccccctgcctagagcactccctaaggctgtccctccctcggtcccaccactgggctcagataaggaggcgtggccaacagacacagagtcctataaaggtggtggtgccttcaccctaaccctgaaggtggtagttcttggagcttccccgatcctccctagggtcctagagaacactcttcacttaagctt

(19) United States
(12) Patent Application Publication
(10) Pub. No.: US 2005/0277142 A1
(43) Pub. Date: Dec. 15, 2005

(54) Recombinant cell line for the detection of dioxin-like compounds based on the expression of luciferase gene

(75) Inventors: Wei-Shan Lee, Niausung Shiang (TW); Hsin-Yu Lee, Taipei City (TW); Guo-Ping Chang-Chien, Niausung Shiang (TW); Yi-Wei Huang, Taipei City (TW); Kuan-Yu Lu, Niausung Shiang (TW)

Correspondence Address: FOLEY AND LARDNER LLP;SUITE 500, 3000 K STREET NW, WASHINGTON, DC, 20007, US

(73) Assignee: Cheng-Shiu University

(21) Appl. No.: 11/124,386

(22) Filed: May 9, 2005

(30) Foreign Application Priority Data
Jun. 14, 2004 (TW) 093117045

(51) Int. Cl. C12Q 1/68; C12Q 1/00; C07H 21/04; C12N 5/06; C07K 14/705

(52) U.S. Cl. 435/6; 536/23.5; 435/4; 435/69.1; 435/354; 435/320.1; 530/350

(57) ABSTRACT

Disclosed herein are recombinant cell lines harboring therein an exogenous luciferase gene, in particular transfected mouse hepatoma cell lines Hepa1c1c7-M4P2 and Hepa1c1c7-MP, in which a DNA fragment having nucleotide sequences “cacgc” and “gcgtg” that may be specifically present in dioxin-response elements is located upstream of the luciferase gene, the DNA fragment being designed based on the promoter region and a part of the front sequence of the structure gene of Mus musculus CYP1A1 gene. As such, the recombinant cell lines and the subcultured offsprings thereof can be used via bioassay for the detection of dioxin-like compounds present in a sample collected from a natural environment, including: soil; water sources; edible meats, fishes, vegetables and fruits and the like; and flues, bottom ashes, sludge or the like in plants or incinerators.

### CROSS-REFERENCE TO RELATED APPLICATION

This application claims priority of Taiwanese Application No. 093117045, filed on Jun. 14, 2004.

### BACKGROUND OF THE INVENTION

1. Field of the Invention

The invention relates mainly to recombinant cell lines harboring therein an exogenous luciferase gene, in particular transfected mouse hepatoma cell lines Hepa1c1c7-M4P2 and Hepa1c1c7-MP, in which a DNA fragment having nucleotide sequences “cacgc” and “gcgtg” that may be specifically present in dioxin-response elements is located upstream of the luciferase gene, the DNA fragment being designed based on the promoter region and a part of the front sequence of the structure gene of Mus musculus CYP1A1 gene. As such, the recombinant cell lines and the subcultured offsprings thereof can be used via bioassay for the detection of dioxin-like compounds present in a sample collected from a natural environment (for example: soil; water sources; edible meats, fishes, vegetables, fruits and the like; and flues, bottom ashes, sludge or the like in plants or incinerators).

2. Description of the Related Art

Due to the over-development of civilization, many tangible and non-tangible pollutants invade the society of human beings living in the civilized world. Amongst the various pollutants, dioxin is a chemical that is currently considered to be most toxic to humans and animals, and thus being dubbed as the “toxin of the century.” Studies in the recent two decades have revealed that dioxin not only affects human reproduction, obstructs growth development, damages the immune system and interferes with normal hormone secretion, it also has carcinogenicity. However, dioxin is a substance that is difficult to decompose, and it will continue to accumulate and exist in the natural environment and in organisms, thereby posing a serious latent threat to the entire environment.

Particularly, since dioxin is relatively stable under ordinary conditions, even in a hot, acid or alkaline environment, when it is released into the environment in minute amounts and enters the food chain, it will increase in concentration as it climbs up the food chain. The chemical structure of dioxin is similar to the structure of human hormones (E. L. Gregoraszczuk, Cad Saude Publica., March-April, 2002, 18 (2): 453-62). Therefore, after being uptaken into the human body via the food chain, dioxin will act as a “pseudo-hormone” to result in hormone-like functions, whilst affecting the hormone content in the human body, both of which will interfere with the endogenous endocrine mechanism of the human body, and will seriously affect the metabolism of the human body and interfere with the balance of hormones, thereby resulting in damage to the body's functions.

In addition, dioxin has been proven to be a potent tumor promoter that elicits animal carcinogenicity, including increasing the incidence of hepatoma, lung tumor and skin tumor in rats. The Environmental Protection Association in the United States (US-EPA) and the World Health Organization (WHO) have classified dioxin as a possible human carcinogen. In 1997, the International Agency for Research on Cancer (IARC) classified 2,3,7,8-TCDD, the most toxic dioxin, as a “Class 1 known human carcinogen” (D. B. McGregor et al. (1998), Environ Health Perspect., April; 106 Suppl 2:755-60). Therefore, how to detect this environmental pollutant within a shortest time using an effective method has become an important direction of investigation to relevant researchers.

Dioxin is a general term for a group of compounds that are formed of two benzene rings joined by one or two oxygen atoms. These compounds belong to a group of halogenated aromatic hydrocarbons (HAHs). Dioxin can be divided into two series of coplanar tricyclic compounds, which are: (1) polychlorinated dibenzo-p-dioxins (PCDDs)—this group of compounds has a general chemical formula of C₁₂H₈₋ₙO₂Clₙ, wherein n=1˜8; and (2) polychlorinated dibenzofurans (PCDFs)—this group of compounds has a general chemical formula of C₁₂H₈₋ₙOClₙ, wherein n=1˜8. When the eight substitution positions on the benzene rings are attached with different numbers of chlorine atoms, there will be present 75 isomers for PCDDs and 135 isomers for PCDFs. Of these 210 dioxin isomers, 2,3,7,8-tetrachloro-dibenzo-p-dioxin (2,3,7,8-TCDD) has the highest toxicity and, thus, is dubbed as the “toxin of the century.” PCDDs and PCDFs are aromatic compounds having an almost planar configuration. These two groups of compounds possess similar physical and chemical properties, and likewise have a very stable organic molecular structure, as well as characteristics of high melting points, high boiling points, and difficulty to degrade. Therefore, these compounds are found to exist in various environmental media (such as air, soil, water, and food)(W. Parzefall (2002), Food Chem Toxicol., 40(8):1185-9). Taking 2,3,7,8-TCDD, which has a molecular weight of 322, as an example, decomposition of the same can be achieved only when a high-temperature burning of higher than 850° C. is conducted. As regards volatility, it has a vapor pressure of 7.4×10⁻¹ mmHg at 25° C. In addition, dioxin has a water solubility of 19.3 ng/L, thus being lipophilic, and is relatively stable under normal conditions. Dioxin is extremely stable to heat, acid and alkaline, and its chemical properties will not change unless exposed to isooctane and ultraviolet light.

Given the aforementioned characteristics, dioxin volatilizes very slowly from either water or soil. With reference to the environmental degradation properties thereof, in a simulated aquatic field test, only 5 out of 100 microorganisms can degrade TCDD. Therefore, once produced in the natural environment, dioxin can hardly be degraded and will continue to accumulate in the natural environment.

The measuring unit of dioxin is normally represented by the detected toxicity equivalent values of a medium under test. Since dioxin includes 75 isomers each of which has a different level of toxicity, the responses of test animals thereto vary in a wide range. In addition, the dioxin-like compounds in the environment to which they are exposed in general are a mixture of a variety of different compounds rather than single compounds.

Therefore, in regard to the approaches for the detection of dioxin, many scholars have advocated the viewpoint of the so-called toxicity equivalent coefficients and, based on the degree of sensitivity of aryl hydrocarbon receptor (AHR) to each single compound and in accordance with the relative toxicity influence of the compounds on substances, established the International Toxicity Equivalency Factors (I-TEF), in which the most toxic 2,3,7,8-TCDD was set to have a factor of 1. This 1-TEF value has become the standardized toxicity equivalence factor jointly put forward by the WHO European Center for Environment & Health and the International Programme on Chemical Safety (PCS)(S. H. Safe (1990), Crit. Rev. Toxicol., 21 (1):51-88).

The use of TEF values is based on the following presumptions: (1) the toxic actions of all the dioxin-like compounds were mediated by AHR; (2) the total toxicity of dioxin-like compounds is the summation of the respective toxicities of individual compounds; and (3) the toxic actions of most of the dioxin-like compounds are evaluated only by the value of TEF. Accordingly, the toxicity equivalent of 2,3,7,8-TCDD is calculated as follows:
TEQ=TEF×true concentration

Internationally, the toxicity equivalent values of dioxin-like compounds are expressed in terms of I-TEQ (International Toxic Equivalency). Dioxin concentration in the air may be expressed in ng-TEQ/Nm³, whereas the dioxin concentration in soil may be expressed in pg-TEQ/g. If only the amount of dioxin contained in a medium is to be calculated, the measurement thereof can be expressed in terms of weight unit commonly used in the art, such as pg/g, ppt, ppb, etc. For instance, the unit “pg/kg-bw” refers to the amount of dioxin in pg per kilogram of body weight. The WHO suggests that a tolerable daily intake of dioxin for each person is 1˜4 pg/kg-bw. For an adult of 60 kg, for instance, the highest tolerable daily intake of dioxin will be 240 pg.

In the past two decades, chemical methods for detecting dioxin have been well developed. These methods mainly include the use of gas chromatography in combination with electron capture detection or mass spectrometry. The steps of analysis primarily include: sampling, extraction, purification, separation, and quantification (C. Rappe (1991), IARC Sci Publ., 108:1-426; De Jong APJM and AKD. Liem (1993), Trands Anal Chem., 12:115-124). Chemical detection methods can be used to analyze the concentrations of all the components of dioxin with precision. However, these methods are unsuitable for use in daily routine detections as the entire detection process thereof costs too much time and money.

Later on, there has been developed in the art a kind of immunochemical method-based enzyme-linked immunosorbent assay (ELISA) (Zajicek J L, Application of enzyme-linked immunosorbent assay (ELISA) for measurement of polychlorinated biphenyls from hydrophobic solutions: extracts of fish and dialysates of semipermeable membrane devices. In: Van Emon J M et al., editors. Environmental immunochemical methods, chapter 26. American Chemical Society, 1996, pp. 307-325). This technique is based on the existence of an excellent specificity and affinity between antibodies and their corresponding antigens. Therefore, haptens specific for dioxin chemical structures can be used to produce dioxin-specific antibodies (Y. Sugawara et al. (1998), Anal Chem., 70 (6): 1092-1099), or antibodies capable of specifically identifying AHR that can be bound by dioxin to thereby identify the concentration of exogenous dioxin by detecting the extent of converted AHR (A. Poland et al. (1991), Mol Pharmacol., 39 (4): 435). However, such detection methods are quite complicated in experimental operation.

In recent years, a biosensor technique has been established (S. Bender et al. (1998), Environ Sci Technol., 32:788-797), in which antibodies are used in combination with a suitable measuring system to constitute an immunoreactive biosensor, so that the antigen concentration of a sample can be quantified by measuring the mass variations during the reaction process. For instance, the antibodies are effectively immobilized onto a gold electrode's surface by chemical bonding, and the antigens present in a sample are bound to the immobilized antibodies due to affinity binding, followed by measurement using an optical (optic-fiber and surface plasma resonance) or oscillating crystal (quartz oscillating crystals and surface acoustic wave devices) signal conversion system, so that the antigen concentration of the sample can be effectively quantified. In addition, the antibodies can be subjected to a special treatment, so that their binding with dioxin will result in the emission of an electrochemical signal, the measurement of which is recorded so as to determine the quantity of the dioxin. However, the techniques in this respect have yet to reach a mature stage and, hence, they have not been widely adopted so far.

In order to develop a faster and more convenient detection method of dioxin, and with people's growing awareness of the carcinogenic mechanisms of dioxin in organisms, biological response-based bioassays are receiving more and more attention as time goes by.

When an organism is exposed to an environment containing dioxin, many biological and physiological changes may come about in its body. The existence of dioxin can then be determined by observing these changes. Amongst the toxicity mechanisms of dioxin, the AHR pathway has been most extensively investigated (O. Hankinson (1995), Annu Rev Pharmacol Toxicol., 35:307-40).

After dioxin has entered a cell, it will enter into the cell's nucleus via a series of messages transmitted along the AHR pathway and will ultimately be brought to the dioxin response elements (DREs) due to its binding with the AHR nuclear translocator (ARNT) to thereby modulate gene action. In this aspect, the cytochrome P450 1A (CYPLA) gene is a well-known “in vivo biomarker,” and extensive expression thereof can be induced in human or mouse cells via the AHR pathway. (M. Merchant et al. (1992), Arch Biochem Biophys., 298 (2): 389-94; J. P. Vanden-Heuvel et al. (1993), Carcinogenesis., 14: 2003-2006; Denison M S, Withlock J P Jr. (1995), Journal of Biological Chemistry, 1995; 270 (31):18175-8).

The DREs present in genes of many different cells have been reported (Z.-W. Lai et al. (1996), Chemico-Biological Interaction, 100:97-112), and said DREs carry certain specific nucleotide sequences (such as cacgc or gcgtg), which play an important role in the identification of dioxin (J. Corchero et al. (2001), Pharmacogenetics., 11:1-6).

Amongst bioassay methodologies, CALUX (chemical-activated luciferase expression) assay is one that has received considerable attention in recent years. Its major technical concept is to ligate a reporter gene to the downstream of the DREs.

In 1993, Hans Postlind constructed two human CYP1-luciferase plasmids. He cleaved the promoter and 5′-flanking sequence segments from upstream of the human CYP1 gene, and cloned them respectively into plasmids carrying a luciferase gene. The constructed recombinant plasmids were subsequently transferred into the human 101L cells. Thereafter, different concentrations of TCDD were added to stimulate the transformed cells, and the expressed luciferase activity was measured. Therefore, the concentration of dioxin could be determined based on the measured fluorescence intensity (H. Postlind et al. (1993), Toxicol Appl Pharmacol., 118 (2):255-262).

All in all, amongst the current dioxin detection methods, chemical analytical methods are the most accurate, but they bear the drawbacks of complicated operating process, long detection time and high cost. With respect to enzyme immunosorbent assays that are based on immunochemical methods, they have the advantages of specificity and sensitivity of bioassays, but they are still quite complicated in terms of operation. Regarding biosensors that are constructed based on immunoassays, they have yet to reach maturation in terms of the overall technical level thereof and are therefore not widely used. Furthermore, as for bioassays, aside from having toxicological specificity, they are more time-saving and less costly as compared to chemical methods, can detect the TEQ of dioxin-like compounds with sensitivity and specificity, and are suitable for the detection of a large number of environmental samples. Therefore, developing a recombinant cell line that is suitable for the detection of dioxin-like compounds by bioassay has become an investigation direction endeavored by relevant researchers in the art.

### SUMMARY OF THE INVENTION

Therefore, according to a first aspect, this invention provides a primer pair for the cloning of dioxin-responsive DNA fragments, comprising a forward primer and a reverse primer, the forward primer consisting essentially of a nucleotide sequence selected from nucleotide sequences as shown in SEQ ID NO:1 and SEQ ID NO:3, and the reverse primer consisting essentially of a nucleotide sequence selected from nucleotide sequences as shown in SEQ ID NO:2 and SEQ ID NO:4:

| | |
|---|---|
| Forward primer 1 | |
| 5′-cagagagcacctgcaaaaca-3′ | (SEQ ID NO:1) |
| Reverse primer 1 | |
| 5′-ggctacaaagggtgatgctt-3′ | (SEQ ID NO:2) |
| Forward primer 2 | |
| 5′-ctcgaggagggcaggtgaaggtgttag-3′ | (SEQ ID NO:3) |
| Reverse primer 2 | |
| 5′-aagcttaagtgaagagtgttctctagg-3′. | (SEQ ID NO:4) |

Accordingly, when conducting a PCR reaction using a CYP1A1 gene sequence as a template and the primer pair according to this invention, a DNA fragment carrying nucleotide sequences (cacgc and gcgtg) that may be specifically present in dioxin-response elements (DREs) may be obtained.

The DNA fragment may be incorporated into a vector having a reporter gene (e.g., a luciferase gene). Therefore, according to a second aspect, this invention provides a recombinant vector which includes a reporter gene, and a DNA fragment as described above which is located upstream of the reporter gene.

The recombinant vector can then be used to transform a selected host cell. Therefore, according to a third aspect, this invention provides a recombinant cell line, which is formed by transforming a host cell using a recombinant vector as described above.

The recombinant cell line according to this invention and the sub-cultured offsprings thereof can be employed in a bioassay for the detection of dioxin-like compounds in an environmental sample, in which a sample containing dioxin-like compounds is contacted with the recombinant cell line that has been cultured for a period of time, so that the reporter gene carried by the recombinant vector harbored within the recombinant cell line is induced to express a gene product. The gene product (e.g., a luciferase) thus produced will generate a detectable event (e.g., light intensity). Said detectable event can then act as an index for the qualitative and quantitative analysis of the presence of dioxin-like compounds.

### BRIEF DESCRIPTION OF THE DRAWINGS

The above and other objectives, features and advantages of the present invention will become apparent in the following detailed description of the preferred embodiment with reference to the accompanying drawings, of which:

FIG. 1 shows the construction of a cloning vector yT&A (2728 bp), which contains an ampicillin resistance gene (AP), a T₇ promoter, a β-galactosidase gene (lacZ), and multiple cloning sites (MCS);

FIG. 2 shows the construction of a pGL2-promoter vector (5790 bp), which contains an ampicillin resistance gene (Amp^{r}), a SV40 promter, a luciferase gene (luc), and multiple cloning sites;

FIG. 3 shows the construction of a pGL3-basic vector (4818 bp), which contains an ampicillin resistance gene (Amp^{r}), a luciferase gene (luc⁺), and multiple cloning sites;

FIG. 4 shows the nucleotide sequence of a PCR product that was amplified from a PCR reaction conducted by using chromosomal DNAs isolated and purified from mouse macrophage cell J774A.1 as a template, and the forward primer 1 and the reverse primer 1 according to this invention, wherein the PCR product has a total length of 479 bp, and contains seven nucleotide sequences which may be specifically present in dioxin-response elements (DREs), i.e. cacgc and gcgtg (the framed portions);

FIG. 5 shows the nucleotide sequence of a PCR product that was amplified from a PCR reaction conducted by using chromosomal DNAs isolated and purified from mouse macrophage cell J774A.1 as a template, and the forward primer 2 and the reverse primer 2 according to this invention, wherein the PCR product has a total length of 1503 bp, and contains twelve nucleotide sequences which may be specifically present in dioxin-response elements (DREs), i.e. cacgc and gcgtg (the framed portions);

FIG. 6 shows the light intensity results detected after the cloned mouse hepatoma cell line Hepa1c1c7-M4P2 according to this invention had been treated with 100 pM dioxin (2,3,7,8-TCDD) for different periods of time (5, 10, 15, and 20 hours);

FIG. 7 shows the light intensity results detected after the cloned mouse hepatoma cell line Hepa1c1c7-MP according to this invention had been treated with 100 pM dioxin (2,3,7,8-TCDD) for different periods of time (5, 10, 15, and 20 hours);

FIG. 8 shows the light intensity results detected after the cloned mouse hepatoma cell line Hepa1c1c7-M4P2 according to this invention had been treated with different concentrations of dioxin (2,3,7,8-TCDD) for a period of 15 hours;

FIG. 9 shows the light intensity results detected after the cloned mouse hepatoma cell line Hepa1c1c7-MP according to this invention had been treated with different concentrations of dioxin (2,3,7,8-TCDD) for a period of 15 hours;

FIG. 10 shows the light intensity results detected after the cloned mouse hepatoma cell line Hepa1c1c7-M4P2 according to this invention had been treated with different concentrations of dioxin, two concentrations of standard samples S₁ and S₂, and a fly ash sample 29 for a period of 15 hours; and

FIG. 11 shows the light intensity results detected after the cloned mouse hepatoma cell line Hepa1c1c7-MP according to this invention had been treated with different concentrations of dioxin (2,3,7,8-TCDD), two concentrations of standard samples S₁ and S₂, and the fly ash sample 29 for a period of 15 hours.

### DETAILED DESCRIPTION OF THE PREFERRED EMBODIMENTS

Previous researches reveal that, when dioxin enters a cell, it will bind to an aromatic hydrocarbon receptor (AHR) carrying two heat shock proteins 90 (HSP90) to form a complex. The complex will disintegrate after it enters the cell's nucleus, and will ultimately be transmitted to DNA via the actions of a series of downstream molecules to bind to the dioxin-response elements (DREs) present in the DNA, so that genes that may be modulated by the dioxin-response elements are extensively expressed, thereby affecting the physiological system of the entire organism. Aiming at the feature of this transmission path, the applicants attempted to construct a recombinant vector including a dioxin-response element (DRE) and a reporter gene, and to transfer the thus-formed recombinant vector into a competent host cell so as to construct a recombinant cell line carrying the recombinant vector.

The recombinant cell line thus obtained can be used in a bioassay for detecting dioxin-like compounds in a sample obtained from a natural environment. Initially, the recombinant cell line was treated with different concentrations of dioxin standards so as to establish the correlation between dioxin concentration and the expression ability of the reporter gene carried by the recombinant vector harbored within the recombinant cell line.

Thereafter, an original sample probably containing dioxin-like compounds was obtained from a natural environment (e.g., soil; water sources; edible meats, fishes, vegetables, fruits and so forth; and flues, bottom ashes, sludge or the like from factories or incinerators), and was subjected to appropriate treatments according to the various preliminary treatments to be described below, so as to obtain an analyte under test.

Subsequently, the analyte was caused to contact the recombinant cell line for a period of time, and the expression result of the reporter gene carried by the recombinant vector harbored within the recombinant cell line was detected. The thus-obtained detection result was subjected to comparison with the above-established correlation between dioxin concentration and the expression ability of the reporter gene, so that the quantity of dioxin contained in the original sample could be assessed.

According to this invention, the CYP1A1 gene is a source suitable for finding useful dioxin-response elements (DRE). Therefore, according to the nucleotide sequence deposited under accession number AF210905 (including Mus musculus CYP1A1 gene, promoter region and partial sequence) and the nucleotide sequence deposited under accession number X01681 (Mus musculus CYP1A1 gene) as recorded on the NCBI website, the applicants have designed the following two primer pairs directed to the dioxin-response elements in the Mus musculus CYP1A1 gene sequence:

| | |
|---|---|
| Forward primer 1 | |
| 5′-cagagagcacctgcaaaaca-3′ | (SEQ ID NO:1) |
| Reverse primer 1 | |
| 5′-ggctacaaagggtgatgctt-3′ | (SEQ ID NO:2) |
| Forward primer 2 | |
| 5′-ctcgaggagggcaggtgaaggtgttag-3′ | (SEQ ID NO:3) |
| Reverse primer 2 | |
| 5′-aagcttaagtgaagagtgttctctagg-3′ | (SEQ ID NO:4) |

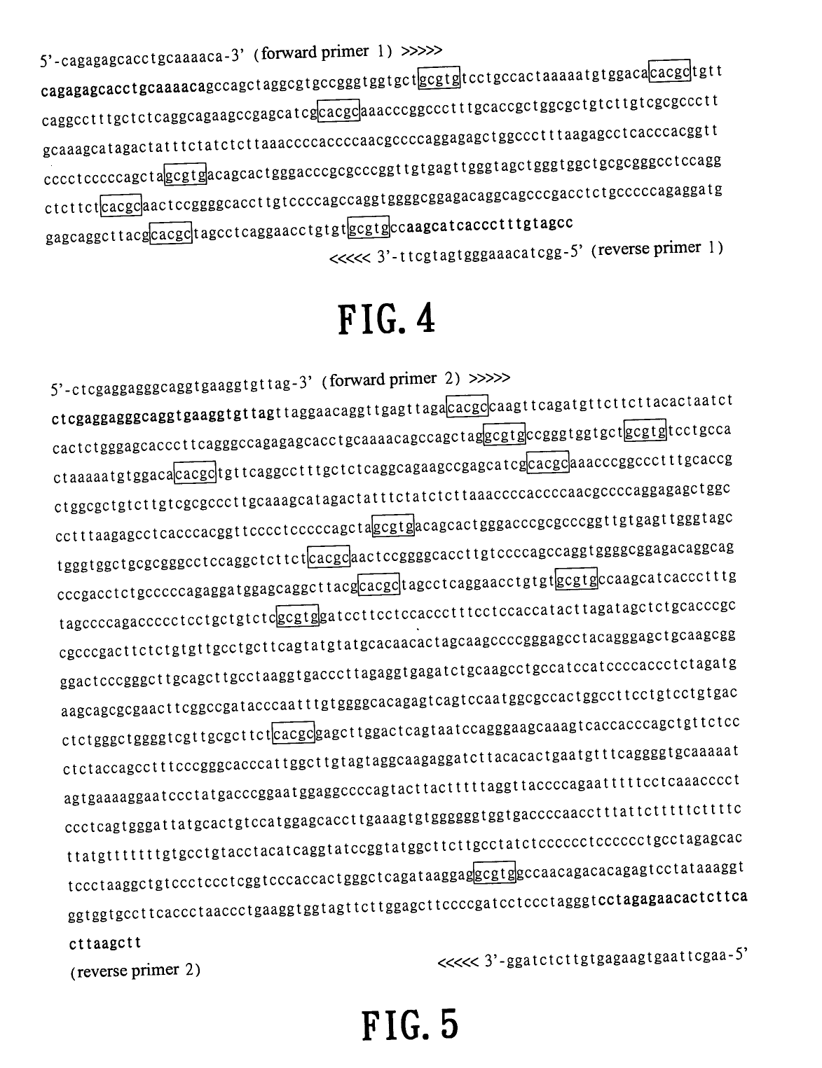

Accordingly, when the Mus musculus CYP1A1 gene sequence was used as a template, by using the first primer pair which was constituted of the forward primer 1 and the reverse primer 1 to carry out polymerase chain reaction (PCR), a PCR product of 479 bp (SEQ ID NO.:5) could be obtained (see FIG. 4), whereas, by using the second primer pair which was constituted of the forward primer 2 and the reverse primer 2 to carry out PCR, a PCR product of 1503 bp (SEQ ID NO:6) could be obtained (see FIG. 5). These two PCR products were found to respectively carry 7 and 12 nucleotide sequences that might be specifically present in the dioxin-response elements (i.e. cacgc and gcgtg). Thus, this invention utilizes these two PCR products to further construct the desired recombinant vectors.

According to this invention, intermediate cells (e.g., Escherichia coli cells) may be used in the cloning process of recombinant vectors so as to extensively amplify the aforesaid two PCR products. One can readily implement the experimental methodologies and materials involved in the cloning process by referring to textbooks well known in the art. For instance, reference can be made to Sambrook J, Russell DW (2001) Molecular Cloning: a Laboratory Manual, 3rd ed. Cold Spring Harbor Laboratory Press, New York.

Vectors suitable for the insertion of either one of the aforesaid PCR products include a marker gene or reporter gene for screening the vectors under appropriate conditions.

Marker genes suitable for use in this invention may include, for example, dihydrofolate reductase gene and G418 or neomycin resistance gene useful in eukaryotic cell cultures, and ampicillin, streptomycine, tetracycline or kanamycine resistance gene useful in E. coli and other bacterial cultures.

Vectors suitable for use in this invention may include other expression control elements, such as a transcription starting site, a transcription termination site, a ribosome binding site, an RNA splicing site, a polyadenylation site, a translation termination site, and multiple cloning sites (MCS). Vectors suitable for use in this invention may further include additional regulatory elements, such as transcription/translation enhancer sequences. Vectors suitable for use in this invention may further include a nucleic acid sequence encoding a secretion signal. These sequences are well known to those skilled in the art.

In a preferred embodiment of this invention, a vector carrying a luciferase gene (luc/luc+) acting as the reporter gene was used, and said vector includes, but is not limited to, pGL2-promoter vector and pGL3-basic vector.

In a preferred embodiment of this invention, the PCR product of 479 bp was incorporated into a pGL2-promoter vector after undergoing proliferation treatment of intermediate cells, to thereby obtain a recombinant vector M4P2.

In another preferred embodiment of this invention, the PCR product of 1503 bp was incorporated into a pGL3-basic vector after undergoing proliferation treatment of intermediate cells, to thereby obtain a recombinant vector MP.

The aforesaid two recombinant vectors may then be transferred into a competent host cell so as to generate desired recombinant cell lines. In a preferred embodiment of this invention, the competent host cell is mouse hepatoma cell Hepa-1C1C7. After transfecting the mouse hepatoma cell Hepa-1C1C7 using the recombinant vector M4P2 and the recombinant vector MP, respectively, mouse hepatoma cloned cell line Hepa1c1c7-M4P2 and mouse hepatoma cloned cell line Hepa1c1c7-MP were obtained.

The mouse hepatoma cloned cell lines Hepa1c1c7-Mp and Hepa1c1c7-M4P2 were successively deposited in the Biosource Collection and Research Center of the Food Industry Research and Development Institute (BCRC of FIRDI)(331 Shih-Pin Road, Hsinchu City 300, Taiwan, R.O.C.) on May 7, 2004 and May 14, 2004 under accession numbers BCRC 960207 and BCRC 960208, respectively. These two cell lines were also deposited in the American Type Culture Collection (ATCC, P.O. Box 1549, Manassas, Va. 20108, USA) under the Budapest Treaty on Jun. 18, 2004, and were given ATCC accession numbers PTA-6089 and PTA-6090, respectively.

The applicants further determined the response ability of the aforesaid cell lines with respect to dioxin, in which 2,3,7,8-TCDD was used as the test compound and the test concentrations and reaction times were varied, and successfully established the correlation between the expression ability of the luciferase gene carried by the recombinant vector harbored within either one of the two cell lines and the concentration of dioxin. Therefore, the recombinant cell lines according to this invention or the sub-cultured offsprings thereof can be used via bioassay to qualitatively or quantitatively examine dioxin-like compounds present in a sample collected from a natural environment (e.g., soil; water sources; edible meats, fishes, vegetables, fruits and the like; and flues, bottom ashes, sludge or the like in factories or incinerators).

This invention will be further described by way of the following examples. However, it should be understood that the following examples are intended for the purpose of illustration only and should not be construed as limiting the invention in practice.

### EXAMPLES

General Experimental Methods and Materials:

Concerning the experimental methods and relevant techniques for DNA cloning as employed herein, such as DNA cleavage reaction by restriction enzymes, DNA ligation with T4 DNA ligase, polymerase chain reaction (PCR), agarose gel electrophoresis, sodium dodecyl sulfate-polyacrylamide gel electrophoresis (SDS-PAGE), and plasmid transformation, etc., reference is made to a textbook well known in the art: Sambrook J, Russell D W (2001) Molecular Cloning: a Laboratory Manual, 3rd ed. Cold Spring Harbor Laboratory Press, New York. These techniques can be readily performed by those skilled in the art based on their professional knowledge and experience. For instance, in the following examples, calcium chloride-treated competent cells were used to conduct the plasmid transformation.

The preparation of small amounts of plasmids, the preparation of large amounts of plasmids, and the purification and recovery of DNA fragments were respectively conducted using commercially available purification kits, i.e., Plasmid Miniprep Purification Kit (Bertec Enterprise Co., Ltd.), Plasmid Midiprep kit (VIOGENE), and Gel Elution Kit (VIOGENE).

I. Experimental Materials:

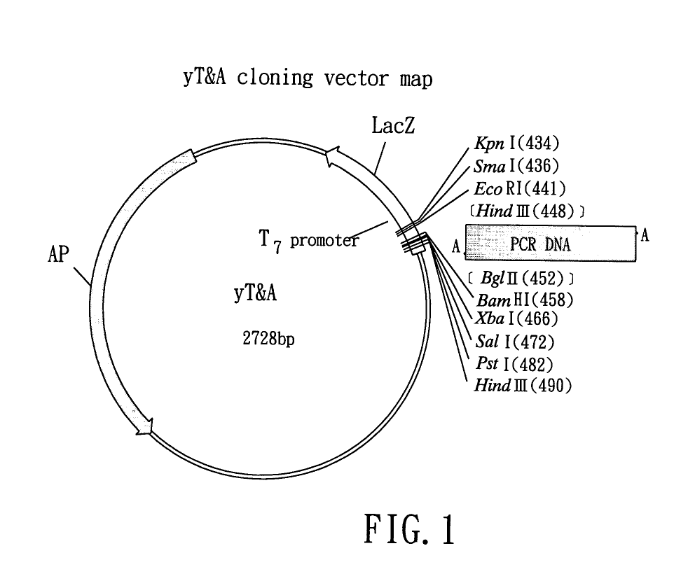

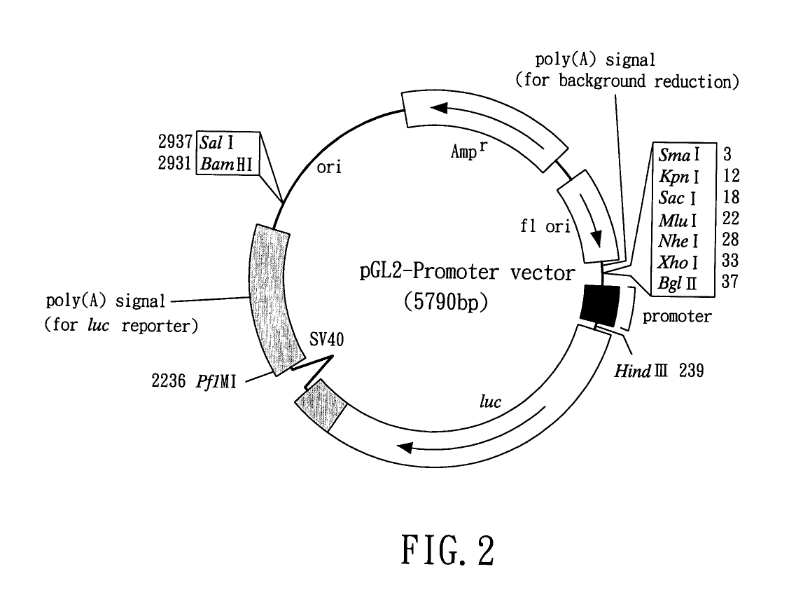

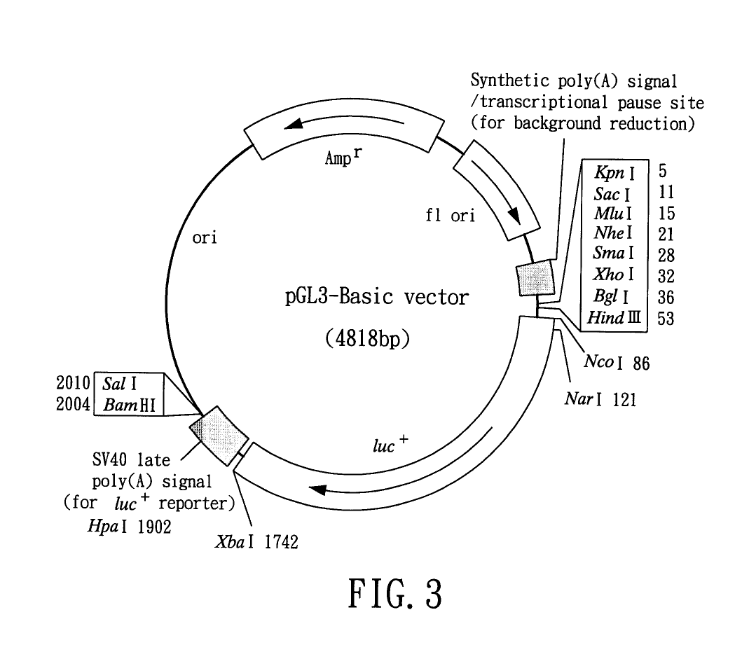

1. Dioxin (2,3,7,8-tetrachlorodibenzo-p-dioxin, (2,3,7,8-TCDD) purchased from Wellington Laboratories, Inc.) solution, the original concentration of which was 10 μg/mL, and was diluted into different concentrations using DMSO prior to experimentation;
   2. Dioxin standards (including 17 dioxin/furan compounds: 2,3,7,8-TCDD, 2,3,7,8-TCDF, 1,2,3,7,8-PeCDD, 1,2,3,7,8-PeCDF, 2,3,4,7,8-PeCDF, 1,2,3,4,7,8-HxCDD, 1,2,3,6,7,8-HxCDD, 1,2,3,7,8,9-HxCDD, 1,2,3,4,7,8-HxCDF, 1,2,3,6,7,8-HxCDF, 1,2,3,7,8,9-HxCDF, 2,3,4,6,7,8-HxCDF, 1,2,3,4,6,7,8-HpCDD, 1,2,3,4,6,7,8-HpCDF, 1,2,3,4,7,8,9-HpCDF, OCDD, OCDF) were provided by the Super Micro Mass Research and Technology Center of Cheng-Shiu University;
3. The following experimental materials were purchased from Protech Technology Enterprise Co., Ltd.: TBE buffer (Tris/borate/EDTA), PBS buffer (NaCl/KCl/Na₂HPO₄/KH₂PO₄, pH7.4), isopropyl-β-D-thiogalactopyranoside (IPTG), phenol/chloroform/isoamyl alcohol, and 5-bromo-4-chloro-3-indoyl-β-D-galactoside (X-Gal);
   4. The following experimental materials were purchased from Yeastern Biotech Co., Ltd.: YEA DNA polymerase, 10× buffer;
5. Culture media: Minimum essential medium (MEM) was purchased from BioWest; Dulbecco's Modified Eagles Medium (DMEM) was purchased from GibcoBRL; Alpha-minimum essential medium (α-MEM) was purchased from BioWest; LB Broth was purchased from Athena Enzyme System Inc.; Nutrition Agar was purchased from Becton Dickson Inc.; Fetal Bovine serum (FBS) was purchased from Hyclone Inc.; and horse serum (HS) was purchased from BioWest;
6. Antibodies for cell culture: ampicillin (A1593) was purchased from Sigma; penicillin-streptomycin was purchased from Invitrogen Inc.; and GENETICIN® (G-418 sulfate) was purchased from GibcoBRL;
7. Restriction enzymes: KpnI and BglII were purchased from New England Biolabs, Ltd. (NEB); HindIII was purchased from Promega; and XhoI was purchased from GibcoBRL;
8. Luciferase Assay System (Cat#1500) was purchased from Promega Cooperation; pcDNA was purchased from Invitrogen Cooperation; GenePORTER™ 2 Transfection Reagent was purchased from GST Inc.; SeaKem LE agarose was purchased from FMC Bioproducts Inc.; trypsin-EDTA was purchased from GibcoBRL; DMSO and EtBr (E8751) were purchased from Sigma; 1 Kb DNA Ladder and loading dye buffer were purchased from Biolabs Inc.;
9. The following experimental materials were purchased from Yeastern Biotech Co., Ltd.: E. coli strain JM109, the culture and storage of which were based on the product specification provided by the supplier; yT&A Cloning Vector Kit, including yT&A cloning vector, ligation buffer A, ligation buffer B, T₄ DNA ligase, in which the construction of the yT&A cloning vector (2728 bp) is shown in FIG. 1 and includes an ampicillin resistance gene (AP), a T₇ promoter, a β-galactosidase gene (lacZ), and multiple cloning sites (MCS); and
10. The following vectors were purchased from Promega Cooperation: pGL2-promoter vector (5790 bp), the construction of which is shown in FIG. 2 and includes an ampicillin resistance gene (Amp^{r}), a SV40 promoter, a luciferase gene (luc), multiple cloning sites, etc.; and pGL3-basic vector (4818 bp), the construction of which is shown in FIG. 3 and includes an ampicillin resistance gene (Amp^{r}), a luciferase gene (luc+), multiple cloning sites, etc.
   II. Cell Lines and Culture Conditions:

The host cells used for transfection were mouse hepatoma cell Hepa-1C1C7 and mouse macrophage cell J774A.1, both of which were purchased from the Biosource Collection and Research Center of the Food Industry Research and Development Institute (BCRC of FIRDI)(331 Shih-Pin Road, Hsinchu City 300, Taiwan, R.O.C.) and were given accession numbers BCRC 60104 and BCRC 60140, respectively. The two cell lines may be sub-cultured according to the following procedures:

1. Hepa-1C1C7 Cells:

The cells were mixed with nucleotide-free Alpha minimum essential medium (α-MEM) supplemented with 10% FBS, and the thus-formed cell suspension was placed into a petri dish to allow cultivation in an incubator with culture conditions set at 37° C. and 5% CO₂. When 80-90% of the bottom area of the petri dish was covered by the cultured cells, the culture solution was removed, and the cells were washed with 3-5 mL PBS buffer (pH 7.4), followed by the addition of 1 mL trypsin-EDTA so as to detach the cells from the bottom of the petri dish. Thereafter, fresh culture solution was added to neutralize trypsin's activity, and the resultant culture solution was repeatedly pipetted using a pipette so as to disperse the cells. Subsequently, the thus-formed cell suspension was dispensed into petri dishes (10 cm in diameter), which were in turn subjected to cultivation in an incubator with culture conditions set at 37° C. and 5% CO₂.

2. J774A.1 Cells:

The cells were added to Dulbecco's Modified Eagles Medium (DMEM), which contained 4 mM L-glutamine and was adjusted to contain 1.5 g/L sodium carbonate and 4.5 g/L glucose and was supplemented with 10% fetal bovine serum, and the thus-formed cell suspension was placed into a petri dish to allow cultivation in an incubator with the culture conditions set at 37° C. and 5% CO₂. When 80-90% of the bottom area of the petri dish was covered by the cultured cells, the culture solution was removed, and the cells were washed with 3 to 5 mL of PBS buffer (pH 7.4). Subsequently, the bottom of the petri dish was slightly scraped using a scraper so as to detach the cells from the bottom of the petri dish. Thereafter, fresh culture solution was added, and the culture solution was repeatedly pipetted using a pipette so as to disperse the cells. The thus-formed cell suspension was then dispensed into petri dishes (10 cm in diameter), which were in turn subjected to cultivation in an incubator with the culture conditions set at 37° C. and 5% CO₂.

III. Preliminary Treatments of Dioxin-Containing Samples From Different Natural Sources:

1. Preliminary Treatment of Environmental Samples:

Environmental samples primarily refer to samples obtained from the following sources: soil; water sources; and flues, fly ashes, bottom ashes and sludge in factories or incinerators.

(1) Extraction Step:

A suitable amount of a sample was weighed and put into a washed thimble filter (Toyo Roshi Kaisha, Ltd.). The thimble filter was put into a soxhlet, followed by the addition of 2 g of anhydrous sodium sulfate. Subsequent to addition of 150 mL of toluene into a 250-mL flat-bottomed flask, the sample was subjected to Soxhlet extraction treatment with refluxing for a period of 24 hours. Thereafter, the toluene extract was concentrated to about 1 mL by concentration under reduced pressure for subsequent treatment.

(2) Acid-Wash Step:

The toluene extract obtained in step (1) was placed into a 24-mL sample flask having a Teflon-lined cap, followed by addition of 7 mL n-hexane, and shaking of the sample flask for about 5 seconds. 4 mL of concentrated sulfuric acid (98%) was then added so as to perform acid wash, and the sample flask was shaken vigorously for about 20 seconds. Thereafter, centrifugation (2000 rpm, 2 minutes) was conducted so as to allow separation of layers. The sulfuric acid layer was collected, whereas the n-hexane layer was subjected to further acid wash using sulfuric acid until the sulfuric acid layer turned white (no more than three times of acid wash). The n-hexane layer was collected in a 50-mL sample flask. Each sulfuric acid layer was further extracted twice using 7 mL of n-hexane. All the n-hexane layers were pooled into the 50-mL sample flask, and were concentrated under reduced pressure to about 1 mL for subsequent treatment.

(3) Purification Step Using a Multi-Layered Silica Gel Column:

After being loaded with glass wool at the tip end thereof, a multi-layer silica gel column (0.5 cm in diameter, 20 cm in length) was packed in sequence with 0.5 g of silica gel, 0.5 g of AgNO₃-silica gel, 0.5 g of silicon gel, 0.5 g of NaOH-silica gel, 0.5 g of silica gel, 5 g of sulfuric acid-silica gel, 0.5 g of silica gel, and 0.5 g of anhydrous sodium sulfate. During packing, these materials were compressed firmly with a glass rod. The packed column was then pre-washed with 30 mL of n-hexane, and the wash solution was discarded.

1 mL of the acid-washed product obtained in step (2) was placed into a pre-washed multi-layer silica gel column. After all of the acid-washed product had entered the column, the sample flask containing the acid-washed product was washed with 1 mL of n-hexane for a total of three times, and the n-hexane wash solutions were placed into the column. Subsequently, the column was eluted twice using 5 mL of n-hexane, followed by eluting the column with 120 mL of n-hexane. The eluate was collected in a 300-mL conical flask and was subjected to nitrogen purge until almost dry. The purified product obtained at this stage contained dioxin compounds, non-planar polychlorinated biphenyl compounds and planar polychlorinated biphenyl compounds.

If it is desired to analyze the total concentrations of the aforesaid three types of compounds contained in the purified product, the purified product can be dissolved in DMSO and then subjected to the bioassay method to be described below so as to determine the total concentration of these compounds.

If it is desired to analyze the respective concentrations of dioxin compounds, non-planar polychlorinated biphenyl compounds and planar polychlorinated biphenyl compounds contained in the purified product, the purified product needs to undergo the following purification steps using an acid aluminum oxide column and an activated carbon column so as to separate dioxin compounds, non-planar polychlorinated biphenyl compounds and planar polychlorinated biphenyl compounds, and the separated compounds can be subjected to concentration analysis using the bioassay method to be described below.

(4) Purification Step Using an Acid Aluminum Oxide Column:

After being loaded with glass wool at the tip end thereof, an acid aluminum oxide column (0.5 cm in diameter, 20 cm in length) was packed in sequence with 1 g of silica gel, 6 g acid of aluminum oxide, 1 g of silica gel, and 1 g of anhydrous sodium sulfate. The packed column was pre-washed with 20 mL of n-hexane, and the wash solution was discarded.

The eluate which was eluted from the multi-layer silica gel column and which was nitrogen-purged to 1 mL in step (3) was placed into the pre-washed acid aluminum column, and was eluted twice using 5 mL of n-hexane, followed by elution of the column using 90 mL of n-hexane. The eluate was collected in a 150-mL conical flask. The eluate obtained at this stage contained non-planar polychlorinated biphenyl compounds.

If it is desired to analyze the concentration of the non-planar polychlorinated biphenyl compounds contained in the eluate, the eluate can be subjected to nitrogen purge until almost dry and then dissolved in DMSO, followed by analyzing the concentration of the compounds using the bioassay method to be described below.

After completion of n-hexane elution, the acid aluminum oxide column was subjected to a further elution using 20 mL of methylene chloride/n-hexane (20/80, v/v). The eluate was collected in a 50-mL sample flask, and was nitrogen-purged slowly until almost dry. The eluate obtained at this stage contained a mixture of dioxin compounds and planar polychlorinated biphenyl compounds. The eluate was dissolved in 1 mL of n-hexane and was loaded into a sample flask so as to conduct the following activated carbon column purification step.

(5) Purification Step Using an Activated Carbon Column:

After being loaded with glass wool at the tip end thereof, an activated carbon/celite column (0.5 cm in diameter, 20 cm in length) was packed in sequence with 0.5 g of silica gel, 0.5 g of activated carbon/celite (18/82, v/v), and 0.5 g of silica gel. During packing, these materials were compressed firmly with a glass rod. Thereafter, the packed column was sequentially pre-washed with methanol, toluene, methylene chloride/methanol/toluene (75/20/5, v/v/v), cyclohexane/methylene chloride (50/50, v/v) and n-hexane, each of 5 mL. The wash solutions were discarded.

1 mL of the n-hexane solution, which was obtained subsequent to the methylene chloride/n-hexane elution in the aforesaid step (4), was placed into the activated carbon/celite column. After all of the n-hexane solution had entered into the column, the sample flask was washed with 1 mL of n-hexane for a total of three times, and the n-hexane wash solutions were further placed into the column.

Subsequently, the activated carbon/celite column was eluted using 2 mL of cyclohexane/methylene chloride (50/50, v/v) for a total of four times, and the column was further eluted twice using 1 mL of methylene chloride/methanol/toluene (75/20/5, v/v/v). All the eluates were combined into a 50-mL sample flask. The eluate solution obtained at this stage contained planar polychlorinated biphenyl compounds.

If it is desired to analyze the concentration of the planar polychlorinated biphenyl compounds, the collected eluate solution can be nitrogen-purged until almost dry and then dissolved in DMSO, followed by analyzing the concentration of the compounds using the bioassay method to be described below.

After completion of the elution, the activated carbon/celite column was subjected to a further elution using 35 mL of toluene, and the eluate was collected in a 150-mL conical flask. The eluate obtained at this stage contained dioxin compounds.

If it is desired to analyze the dioxin compounds alone, the collected toluene eluate can be nitrogen-purged until almost dry and then dissolved in DMSO, followed by analyzing the concentration of the compounds using the bioassay method to be described below.

2. Preliminary Treatment of Plant Samples:

(1) Extraction of Plant Samples:

A suitable amount of a sample was weighed and placed into a 250-mL serum bottle, followed by the addition of 50 mL of n-hexane, 50 mL of methylene chloride, 25 mL of 37.5% HCl, and 25 mL of ultra-pure water, and the serum bottle was shaken for a period of 24 hours. Thereafter, the resultant mixture was poured into a separating funnel, and the serum bottle was washed twice using 50 mL of n-hexane. The washing solution was also poured into the separating funnel. The separating funnel was allowed to stand so as to form an organic upper layer and an aqueous lower layer. The organic upper layer was collected, and the aqueous lower layer was washed twice using 50 mL of n-hexane. Thereafter, the two n-hexane wash solutions were combined with the organic upper layer, and the resultant organic mixture was concentrated under reduced pressure to about 1 mL of concentrate.

Subsequently, the 1-mL concentrate can be subjected to further treatments according to the acid wash step (2), the purification step (3) using a multi-layered silica gel column, the purification step (4) using an acid aluminum oxide column, and the purification step (5) using an activated carbon column as described in the preceding section of “Preliminary treatments of environmental samples.”

3. Preliminary Treatments of Fat-Containing Samples:

(1) Extraction of Fat-Containing Samples:

Fat-containing samples primarily refer to samples obtained from the following sources: meats, feeds, blood and dairy products.

A suitable amount of a sample was weighed and placed into a 250-mL brown flask, to which 150 mL of methylene chloride was added. The brown flask was shaken for a period of 24 hours. Subsequently, the methylene chloride extract was poured into a flask, and the brown flask was washed twice using 50 mL of methylene chloride. All of the wash solutions were poured into the flask, and the resultant mixture was concentrated under reduced pressure to about 1 mL of concentrate.

(2) Detection of Oil Content %:

The 1-mL concentrate obtained in the aforesaid extraction step (1) was passed through a column (0.5 cm in diameter, 20 cm in length) containing 0.5 g of silica gel, and the column was eluted using 50 mL of methylene chloride. The collected eluate was concentrated to dryness to give a fat. The oil content % of the fat-containing sample could be calculated by dividing “the weight of the obtained fat” by “the weight of the fat-containing sample.”

(3) Acid Wash Step:

The fat obtained in the aforesaid step (2) was added into a 50-mL sample flask having a Teflon-lined cap, followed by the addition of 10 mL of n-hexane, and the sample flask was shaken for about 5 seconds. 7 mL of concentrated sulfuric acid (98%) was then added so as to perform acid wash, and the sample flask was shaken vigorously for about 20 seconds. Thereafter, centrifugation (200 rpm, 2 minutes) was conducted to thereby obtain an n-hexane layer and a sulfuric acid layer. The sulfuric acid layer was collected, whereas the n-hexane layer was further acid-washed with sulfuric acid until the sulfuric acid layer turned white (no more than three times of acid wash). The n-hexane layers were collected in a 50-mL sample flask. Each sulfuric acid layer was extracted twice using 7 mL of n-hexane. All the n-hexane layers were collected in the 50-mL sample flask, and were concentrated under reduced pressure to about 1 mL of concentrate for subsequent treatment.

(4) Purification Step Using an Acid Silica Gel Column:

After being loaded with glass wool at the tip end thereof, an acid silica gel column (0.5 cm in diameter, 20 cm in length) was packed in sequence with 20 g of silica gel, 100 g of sulfuric acid silica gel, 20 g of silica gel, and 10 g of anhydrous sodium sulfate. During packing, these materials were compressed firmly with a glass rod. The packed column was pre-washed with 50 mL of n-hexane, and the wash solution was discarded.

The 1-mL concentrate obtained in the aforesaid acid wash step (3) was placed into the pre-washed acid silica gel column. After all of the concentrate had entered into the column, the 50-mL sample flask was washed with 1 mL of n-hexane for a total of three times, and all of the n-hexane wash solutions were placed into the acid silica gel column. Subsequently, the column was eluted twice using 5 mL of n-hexane, followed by elution of the column using 500 mL of n-hexane. The eluate was collected in a 1000-mL conical flask. The collected eluate was nitrogen-purged until almost dry.

Thereafter, the thus-obtained residue can be subjected to further treatments according to the purification step (3) using a multi-layered silica gel column, the purification step (4) using an acid aluminum oxide column, and the purification step (5) using an activated carbon column as described in the preceding section of “Preliminary treatments of environmental samples.”

### Example 1

### Construction of Recombinant Plasmids M4P2 and MP Containing Dioxin-Response Elements

Based on the nucleotide sequence deposited under accession number AF210905 (including Mus musculus CYP1A1 gene, promoter region and partial sequence) and the nucleotide sequence deposited under accession number X01681 (Mus musculus CYP1A1 gene) as recorded on the NCBI website, and by utilizing the software Primer3 Version, the following two primer pairs were designed for the dioxin-response elements (DREs) in the Mus musculus CYP1A1 gene sequence, in which the nucleotide sequences of the primers were synthesized by Protech Technology Enterprise Co., Ltd.:

| | |
|---|---|
| Forward primer 1 | |
| 5′-cagagagcacctgcaaaaca-3′ | (SEQ ID NO: 1) |
| Reverse primer 1 | |
| 5′-ggctacaaagggtgatgctt-3′ | (SEQ ID NO:2) |
| Forward primer 2 | |
| 5′-ctcgaggagggcaggtgaaggtgttag-3′ | (SEQ ID NO:3) |
| Reverse primer 2 | |
| 5′-aagcttaagtgaagagtgttctctagg-3′ | (SEQ ID NO:4) |

wherein the forward primer 2 and the reverse primer 2 were respectively designed to have cutting sites of restriction enzymes XhoI and HindIII (see the underlined portions).

Therefore, when conducting polymerase chain reaction (PCR) using the Mus musculus CYP1A1 gene sequence as a template, a PCR product of 479 bp (SEQ ID NO:5)(see FIG. 4) could be obtained through use of the first primer pair constituted of the forward primer 1 and the reverse primer 1, whereas a PCR product of 1503 bp (SEQ ID NO:6) (see FIG. 5) could be obtained through use of the second primer pair constituted of the forward primer 2 and the reverse primer 2. These two PCR products, which carried nucleotide sequences (cacgc and gcgtg) that might be specifically present in dioxin-response elements, were used in the following experiments to construct recombinant vectors M4P2 and MP.

(A) Construction of Recombinant Vector M4P2:

According to the methods described in the preceding section of “General experimental methods,” chromosomal DNA was isolated and purified from mouse macrophage cells J774A.1, and then used as a template in a PCR reaction using the first primer pair constituted of the forward primer 1 and the reverse primer 1, so that a PCR product of 479 bp was amplified. The PCR product has a nucleotide sequence corresponding to nucleotide residues 210-688 of the nucleotide sequence deposited under accession number AF210905 as recorded on the NCBI website, and contains 7 nucleotide sequences which may be specifically present in the dioxin-response elements (DREs), i.e. cacgc and gcgtg (see the framed portions in FIG. 4).

The amplified 479 bp PCR product was cloned into a yT&A cloning vector (2728 bp, the construction of which is shown in FIG. 1), using a yT&A Cloning Vector Kit (Yeastern Biotech Co., Ltd.), followed by transformation of the resultant recombinant vector into E. coli cells JM109 (Yeastern Biotech Co., Ltd) according to the plasmid transformation method described in the preceding section of “General experimental methods.” Ampicillin-resistant colonies grown from the transformed E. coli cells were screened using solid agar plates containing ampicillin.

Subsequently, plasmids were purified from the screened recombinant ampicillin-resistant E. coli colonies using Plasmid Miniprep Purification Kit (Bertec Enterprise Co., Ltd.), and were used as a template in a PCR reaction using the first primer pair, followed by analyzing the PCR results by agarose gel electrophoresis, whereby a recombinant yT&A cloning vector which contains a DNA fragment that consists of a nucleotide sequence substantially corresponding to that of the 479 bp PCR product was screened.

According to the descriptions set forth in the preceding section of “General experimental methods,” restriction enzymes KpnI/BglII were used to cut the screened recombinant yT&A cloning vector to obtain a KpnI-BglII DNA fragment (0.5 Kb).

Thereafter, the KpnI-BglII DNA fragment was incorporated into a pGL2-promoter vector (5790 bp, the construction of which is shown in FIG. 2, in which a luciferase gene (luc) is present)(Promega Cooperation) cleaved with restriction enzymes KpnI/BglII, so as to obtain a recombinant vector M4P2 which includes a DNA fragment that consists of a nucleotide sequence substantially corresponding to that of the 479 bp PCR product, in which the incorporated KpnI-BglII DNA fragment is located upstream of the luciferase gene (luc) of the pGL2-promoter vector.

According to the plasmid transformation method described in the preceding section of “General experimental methods,” the recombinant vector M4P2 was duplicated extensively via transformation into the E. coli strain JM109. Subsequently, the recombinant vector M4P2 was extracted to conduct PCR according to the operating procedures described above, so as to confirm whether the recombinant vector M4P2 included a DNA fragment corresponding to the 479 bp PCR product. The recombinant vector M4P2 was also sequenced for further confirmation.

The confirmed recombinant vector M4P2 was duplicated extensively via transformation into the E. Coli strain JM109, followed by purification using Plasmid Midiprep Purification Kit. The purified recombinant vector M4P2 was then stored under −20° C. for future use.

(B) Construction of Recombinant Vector MP:

According to the method described in the preceding section of “General experimental methods,” chromosomal DNA was isolated and purified from mouse macrophage cells J774A.1, and then used as a template in a PCR reaction using the second primer pair constituted of the forward primer 2 and the reverse primer 2, so that a PCR product of 1503 bp was amplified. The PCR product has a nucleotide sequence corresponding to nucleotide residues 108-1557 of the nucleotide sequence deposited under accession number AF210905 and nucleotide residues 22-63 of the nucleotide sequence deposited under accession number X01681 as recorded on the NCBI website, and includes 12 nucleotide sequences that may be specifically present in dioxin-response elements (DREs), i.e. cacgc and gcgtg (see the framed portions in FIG. 5).

The amplified 1503 bp PCR product was cloned into a yT&A cloning vector using yT&A Cloning Vector Kit (Yeastern Biotech Co., Ltd.), followed by transformation of the resultant recombinant vector into E. coli cells JM109 according to the plasmid transformation method described in the preceding section of “General experimental methods.” Ampicillin-resistant colonies grown from the transformed E. coli cells were screened using solid agar plates containing ampicillin.

Plasmids were purified from the screened recombinant ampicillin-resistant E. coli colonies using Plasmid Miniprep Purification Kit (Bertec Enterprise Co., Ltd.), and were used as a template in a PCR reaction using the second primer pair, followed by analyzing the PCR results by agarose gel electrophoresis, whereby a recombinant yT&A cloning vector including a DNA fragment consisting of a nucleotide sequence substantially corresponding to that of the 1503 bp PCR product was screened.

According to the description in the preceding section of “General experimental methods,” the screened recombinant yT&A cloning vector was cut using restriction enzymes HindIII/XhoI so as to obtain a HindIII-XhoI DNA fragment (about 1.5 Kb). Thereafter, the HindIII-XhoI DNA fragment was sub-cloned into a pGL3-basic vector (4818 bp; the construction of which is shown in FIG. 3, in which a luciferase gene (luc⁺) is present)(Promega Cooperation) cleaved with restriction enzymes HindIII/XhoI, so as to obtain a recombinant vector MP which includes a DNA fragment that consists of a nucleotide sequence substantially corresponding to that of the 1503 bp PCR product, in which the HindIII-XhoI DNA fragment is located upstream of the luciferase gene (luc⁺) of the pGL3-basic vector.

According to the plasmid transformation method described in the preceding section of “General experimental methods,” the recombinant vector MP was duplicated extensively via transformation into the E. coli strain JM109. Subsequently, the recombinant vector MP was extracted to conduct PCR according to the operating method described above, so as to confirm whether the recombinant vector MP included a DNA fragment corresponding to the 1503 bp PCR product. The recombinant vector MP was also sequenced for further confirmation.

The confirmed recombinant vector MP was duplicated extensively via transformation into the E. coli strain JM109, followed by purification using Plasmid Midiprep Purification Kit. The purified recombinant vector MP was then stored under −20° C. for future use.

### Example 2

### Transfection of Mouse Hepatoma Cell Hepa-1C1C7 with Recombinant Vectors M4P2 and MP

Hepa-1C1C7 cell line could be transformed by recombinant vector MP or M4P2 according to the following Preparation Procedure A or Preparation Procedure B, in which cultivation of the Hepa-1C1C7 cell line was conducted according to the operating procedure described in the preceding section of “Cell line and culture conditions.”

Preparation Procedure A:

1. 172 μl of basic DMEM and 28 μl of GenePORTER™ 2 Transfection Reagent (GST Inc.) were mixed evenly to form a first solution. In addition, 200 μl of New DNA diluent B (GST Inc.) and 8 μg of DNA (4 μg each of the selected recombinant vector DNA and pcDNA (Invitrogen Cooperation)) were mixed evenly to form a second solution. The first and second solutions were allowed to stand for 5 minutes, respectively. Thereafter, these two solutions were mixed evenly, and the resultant mixture was allowed to stand for 20 minutes;
2. 2×10⁷ cells/mL of Hepa-1C1C7 cells were added to a petri dish of 10 cm², and cultivated in a constant-temperature incubator set at 37° C. and 5% CO₂ for a period of about 18-20 hours, so that 70%˜80% of the bottom area of the petri-dish was covered by the cells. Thereafter, the culture medium was removed, and the cells were washed twice using 1×PBS. Then, 5 mL of fresh basic DMEM was added to the petri-dish;
3. Subsequent to the addition of the mixture prepared in step 1, the petri-dish was placed in a constant-temperature incubator set at 37° C. and 5% CO₂ for a period of 4 hours. Thereafter, a suitable amount of culture medium (DMEM+20% FBS) was added to the petri-dish, and cultivation was continued for a period of 20 hours;
4. The culture medium was removed, and the cells were washed twice using 1×PBS. After removing of the wash solution, the cells were treated with 1 mL of trypsin-EDTA for a period no longer than 5 minutes, followed by addition of 9 mL of culture medium (DMEM+10% FBS). Subsequently, the resultant cell suspension was dispensed into 6-well culture plates for subsequent cultivation;
5. The cells cultured in step 4 were diluted with culture medium (DMEM+10% FBS) to have a cell density of 1 cell/100 μl, and the resultant cell suspension was inoculated into 96-well culture plates (100 μl/well) and incubated overnight;
6. Each well of the 96-well culture plates was screened under microscope to see whether there was only one cell therein, and said cell was transferred to a 6-well culture plate for subsequent cultivation, so as to establish individual clones of transfected cells; and
7. For the cells cultivated in steps 4-6, dioxin induction, which will be described in Example 3 hereinbelow, can be used to detect whether the cells are capable of expressing luciferase gene so as to confirm whether the thus-established cell line indeed carries the selected recombinant vector M4P2 or MP.
   Preparation Procedure B:
1. A first mixture was prepared according to step 1 described in Preparation Procedure A, but 8 μg of pcDNA (Invitrogen Cooperation) was used in the preparation of the second solution;

The subsequent steps 2-3 are identical to steps 2-3 described in connection with the aforesaid Preparation Procedure A. The following steps are then carried out:
4. The culture medium was removed, and a culture medium (90% basic DMEM+10% FBS, supplemented with G-418) containing GENETICIN® was added to the petri-dish to proceed with cultivation for a period of one week;
5. A second mixture was prepared according to step 1 described in connection with Preparation Procedure A, but 8 μg of the selected recombinant vector DNA was used in the preparation of the same;
6. For cells that passed the GENETICIN® screening in step 4, they were treated with the second mixture prepared in step 5, and then subjected to a repeat of steps 2-3 described in Preparation Procedure A, so that the cells were transfected by the selected recombinant vector M4P2 or MP; and
7. A repeat of steps 4-7 of Preparation Procedure A was performed for the cells cultured in step 6.

The applicants used the two recombinant vectors M4P2 and MP established in Example 1 to transfect Hepa-1C1C7 cells to thereby obtain two transfected mouse hepatoma cell lines, i.e., Hepa1c1c7-MP and Hepa1c1c7-M4P2. These two cell lines were respectively deposited in the Biosource Collection and Research Center of the Food Industry Research and Development Institute (BCRC of FIRDI) in Taiwan on May 7, 2004 and May 14, 2004, and were given accession numbers BCRC 960207 and BCRC 960208, respectively. These two cell lines were also deposited in the American Type Collection Center (ATCC, P.O. Box 1549, Manassas, Va. 20108, USA) according to the provisions of the Budapest Treaty on Jun. 18, 2004, and were given accession numbers PTA-6089 and PTA-6090, respectively.

### Example 3

### Establishment of the Correlation of Dioxin Concentration vs. the Light Intensity Caused by Luciferase

A. Expression of Luciferase Gene in Transfected Cells After the Stimulation of Dioxin:

The transfected cells (4×10⁵ cells/well) were placed in a 24 well-culture plate. To each well was added 1 mL of culture medium (DMEM+10% FBS). Thereafter, the culture plate was placed in a constant-temperature incubator set at 37° C. and 5% CO₂ for two hours to permit cell adhesion.

The Dioxin (2,3,7,8-TCDD) stock solution with a concentration of 10 μg/mL was subjected to 10-fold serial dilution. 3.22 μl of each diluted solution was taken and added to each well of the culture plate, such that the final concentrations of dioxin in the experimental groups are 10 nM, 1 nM, 100 pM, 50 pM, 10 pM, 1 pM and 0.1 pM, respectively. Each experimental group was prepared in quadruplicate. The normal control group was not subjected to any treatment, whereas the solvent control group was treated using 3.22 μl of DMSO.

After the transfected cells were treated with dioxin for a predetermined period of time (5 hrs, 10 hrs, 15 hrs and 20 hrs), the culture medium was removed, and 150 μl of 1× cell lysis solution (which was included in the Luciferase Assay System (Cat#1500), Promega Cooperation) was added to each well. Thereafter, the culture plates were shaken gently for about 15-20 minutes so that the cells could be completely dissolved. Subsequently, the solution in each well was moved to a microtube for centrifugation at 4° C. and 12000 rpm for 2 minutes. The thus-obtained supernatant was the lysate of the dioxin-treated transfected cells.

100 μl of the lysate obtained in each group was taken and added to each well of a non-transparent 96-well plate, followed by addition of 50 μl of luciferin (which is included in the Luciferase Assay System, (Cat#1500), Promega Cooperation). Finally, the plate was sent to a Beckman coulter DU640B spectrophotometer (set at PMT:1100; read length:0.5; and Gain:1˜100) for automatic detection of the light intensity within 20 seconds. The experimental results thus obtained were analyzed and plotted using statistical analysis, so as to determine whether the results have statistic significance.

B. Results:

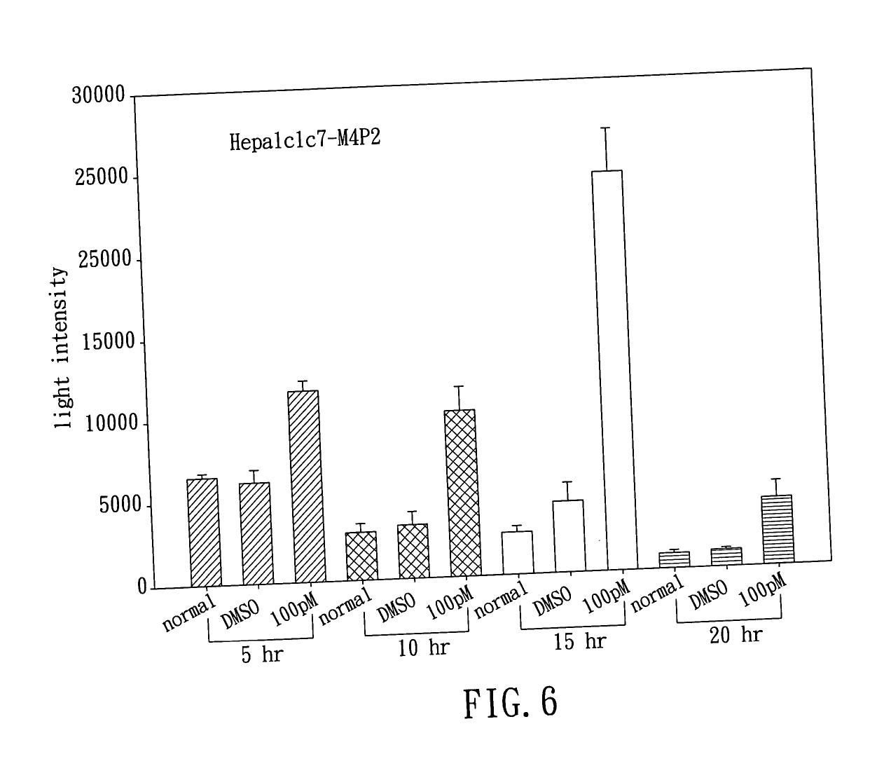

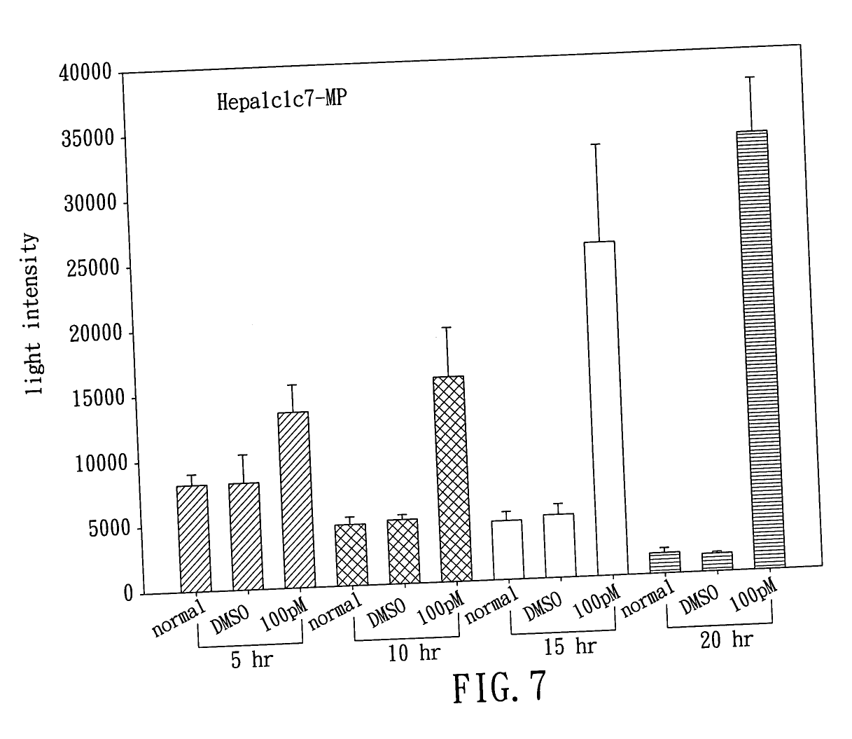

FIGS. 6 and 7 respectively show the light intensity results detected after the transfected mouse hepatoma cell lines Hepa1c1c7-M4P2 and Hepa1c1c7-MP according to this invention were treated with 100 pM of dioxin (2,3,7,8-TCDD) for 5, 10, 15 and 20 hours, in which the detection sensitivity was set at 100 when detecting the light intensity of Hepa1c1c7-M4P2, and was set at 10 when detecting Hepa1c1c7-MP.

The results show that the transfected mouse hepatoma cell line Hepa1c1c7-M4P2 had the most stable and the optimal light intensity result (FIG. 6) after being treated with 100 pM of dioxin (2,3,7,8-TCDD) for 15 hours. In addition, the background values of the normal control group and the DMSO control group were also very low. However, after 20 hours of dioxin treatment, the response of the transfected mouse hepatoma cell line Hepa1c1c7-M4P2 significantly dropped.

As for the transfected mouse hepatoma cell line Hepa1c1c7-MP, it had the most stable and the optimal light intensity result (FIG. 7) after being treated with dioxin for 20 hours, and also exhibited a sufficiently significant response after being treated with dioxin for 15 hours. Besides, the light intensity reading values were so high that it required lowering of the sensitivity of the fluorescence analyzer to 10 in order for them to be completely read.

Based on these experimental results, the dioxin treatment times were all set at 15 hours in the following experiments.

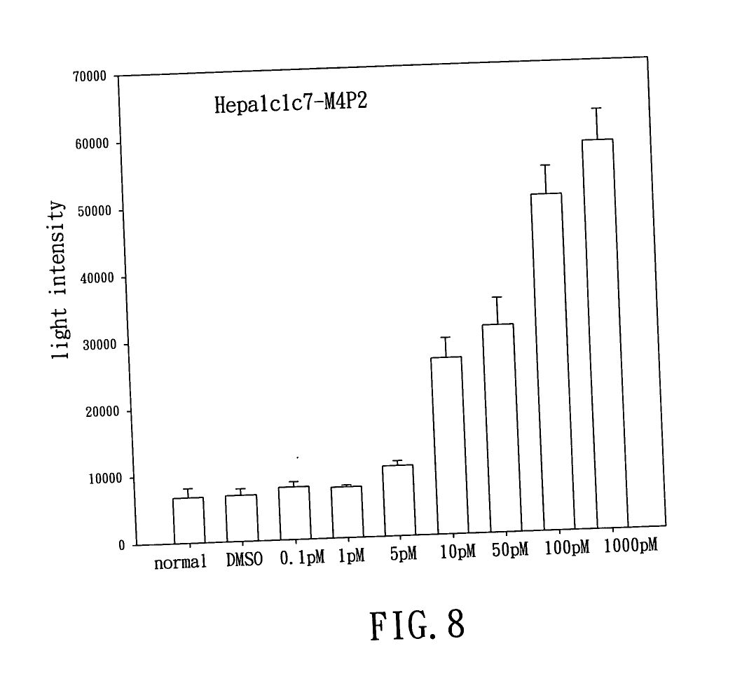

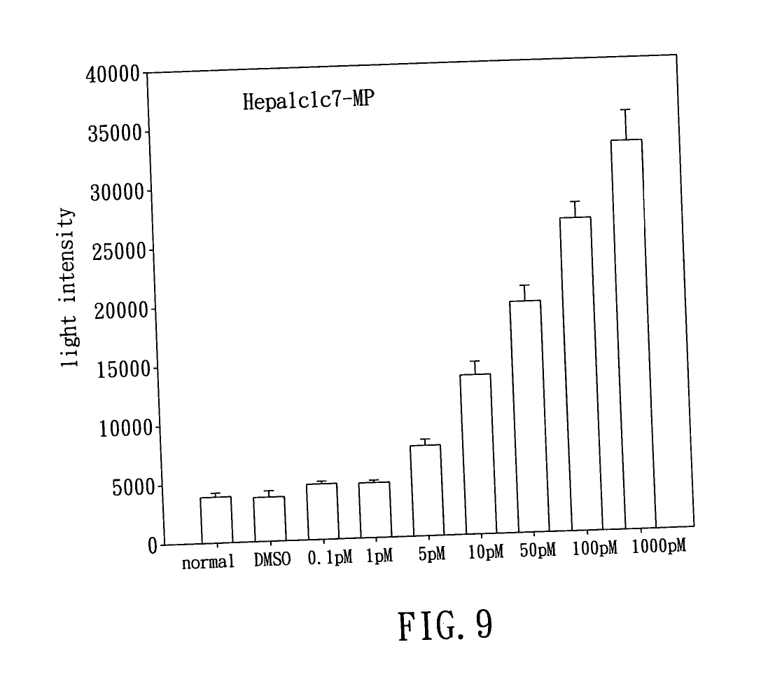

FIGS. 8 and 9 respectively show the results of light intensity detected after the transfected mouse hepatoma cell lines Hepa1c1c7-M4P2 and Hepa1c1c7-MP were treated with dioxin (2,3,7,8-TCDD) of different concentrations (0.1 pM, 1 pM, 5 pM, 10 pM, 50 pM, 100 pM, and 1000 pM) for 15 hours, in which the detection sensitivity was set at 10 when detecting the light intensity of Hepa1c1c7-M4P2, and was set at 1 when detecting Hepa1c1c7-MP. Referring to FIG. 8, when the transfected mouse hepatoma cell line Hepa1c1c7-M4P2 according to this invention was used to detect dioxin (2,3,7,8-TCDD), obvious light intensity changes could be observed by using only 5 pM of 2,3,7,8-TCDD to treat the cells, as compared to the light intensity results of the controls.

As shown in FIG. 9, when the transfected mouse hepatoma cell line Hepa1c1c7-MP according to this invention was used to detect dioxin (2,3,7,8-TCDD), significant light intensity changes could also be observed under the treatment of 5 pM of 2,3,7,8-TCDD, and even stronger than the Hepa1c1c7-M4P2 group treated with the same amount of 2,3,7,8-TCDD. In addition, as compared to the light intensity results of the controls, there exists a very clear positive relationship between dioxin concentrations and the detected light intensities.

In conclusion, for the transfected mouse hepatoma cell lines Hepa1c1c7-M4P2 and Hepa1c1c7-MP according to this invention, the lowest limit of dioxin detection is 5 pM. When taking into account the light intensities of the controls (as background values), the light intensity differences generated by the transfected mouse hepatoma cell line Hepa1c1c7-MP are larger. Therefore, for mouse hepatoma cell Hepa-1C1C7, recombinant vector MP may be a better cloning vector.

### Example 4

### Detection of Dioxin Content in Fly Ash Samples

A. Preparation of Dioxin Standards:

In a suitable container, according to the individual proportions of toxicity coefficients of the 17 dioxin/furan compounds listed in Table 1 below, dioxin standards (S₁ and S₂) with a total concentration of 0.5 ng-TEQ and 0.1 ng-TEQ were formulated using DMSO, followed by nitrogen purging the same to almost dry. The residues were re-dissolved in 128.8 μl of DMSO.

TABLE 1
Formulation of dioxin standards

| | | Stock | | |
|---|---|---|---|---|
| | | solution | | |
| Compound name | I-TEF | (pg/μl) | S₁ (20 μl) | S₂ (100 μl) |
| 2,3,7,8-TCDF | 0.1 | 0.5 | 1 | 5 |
| 1,2,3,7,8-PeCDF | 0.05 | 2.5 | 2.5 | 12.5 |
| 2,3,4,7,8-PeCDF | 0.5 | 2.5 | 25 | 125 |
| 1,2,3,4,7,8-HxCDF | 0.1 | 2.5 | 5 | 25 |
| 1,2,3,6,7,8-HxCDF | 0.1 | 2.5 | 5 | 25 |
| 2,3,4,6,7,8-HxCDF | 0.1 | 2.5 | 5 | 25 |
| 1,2,3,7,8,9-HxCDF | 0.1 | 2.5 | 5 | 25 |
| 1,2,3,4,6,7,8-HpCDF | 0.01 | 2.5 | 0.5 | 2.5 |
| 1,2,3,4,7,8,9-HpCDF | 0.01 | 2.5 | 0.5 | 2.5 |
| OCDF | 0.001 | 5 | 0.1 | 0.5 |
| 2,3,7,8-TCDD | 1 | 0.5 | 10 | 50 |
| 1,2,3,7,8-PeCDD | 0.5 | 2.5 | 25 | 125 |
| 1,2,3,4,7,8-HxCDD | 0.1 | 2.5 | 5 | 25 |
| 1,2,3,6,7,8-HxCDD | 0.1 | 2.5 | 5 | 25 |
| 1,2,3,7,8,9-HxCDD | 0.1 | 2.5 | 5 | 25 |
| 1,2,3,4,6,7,8-HpCDD | 0.01 | 2.5 | 0.5 | 2.5 |
| OCDD | 0.001 | 5 | 0.1 | 0.5 |
| Total amount | | | 0.1 ng-TEQ | 0.5 ng-TEQ |

B. Dioxin Detection:

2 g of a fly ash sample (provided by the Super Micro Mass Research and Technology Center of Cheng-Shiu University) was placed in a beaker, and was subjected to preliminary treatments according to the steps described in the preceding section of “Preliminary treatments of environmental samples” so as to obtain a DMSO-redissolved fly ash sample 29 solution.

According to the descriptions of Example 3, 10-fold serially diluted dioxin (2,3,7,8-TCDD) solutions of 3.22 μl each were used to treat the cells, and the final concentrations of the dioxin in the cell cultures were made to be 10 nM, 1 nM, 100 pM, 50 pM, 10 pM, 1 pM, and 0.1 pM, respectively. Each experimental group was prepared in triplicate. The normal control group was not subjected to any treatment, whereas the solvent control group was treated with 3.22 μl of DMSO.

3.22 μl of each of the fly ash sample solution 29 obtained above and the two standard samples S₁ and S₂, which were formulated in step (A) to have different concentrations, were used to treat cells. Thus, the concentrations of the fly ash sample solution and the two standard samples S₁ and S₂ were diluted 40-fold.

After the cells were treated with dioxin for a period of 15 hours, light intensity was recorded in a manner as described in Example 3, in which the light intensity detection sensitivities were all set at 1.

C. Results:

Table 2 is a table showing the relationship between the concentration and quantity of the dioxin (2,3,7,8-TCDD) in the control standards used for analyzing the samples. Therefore, when estimating the quantity of dioxin-like compounds in the fly ash sample solution and the two standard samples S₁ and S₂, the quantity of dioxin contained in the fly ash sample solution and the two standard samples S₁ and S₂ were determined based on comparisons between the detected light intensity of each sample and the light intensities of the standards, and with reference to the dioxin concentration and quantity relationship shown in Table 2.

TABLE 2
The relationship between the concentration
and the quantity of dioxin

| Volume added | | | | | | | | |
|---|---|---|---|---|---|---|---|---|
| to 1 mL of | Concen- | | | | Toxicity | | | |
| culture medium | tration | | Mass | | equivalency | | ppt | |
| 3.22 μL | 10 | nM | 3220 | pg | 3220 | pg-TEQ | 3220 | ppt |
| 3.22 μL | 5 | nM | 1610 | pg | 1610 | pg-TEQ | 1610 | ppt |
| 3.22 μL | 1 | nM | 322 | pg | 322 | pg-TEQ | 322 | ppt |
| 3.22 μL | 500 | pM | 161 | pg | 161 | pg-TEQ | 161 | ppt |
| 3.22 μL | 100 | pM | 32.2 | pg | 32.2 | pg-TEQ | 32.2 | ppt |
| 3.22 μL | 50 | pM | 16.1 | pg | 16.1 | pg-TEQ | 16.1 | ppt |
| 3.22 μL | 10 | pM | 3.22 | pg | 3.22 | pg-TEQ | 3.22 | ppt |
| 3.22 μL | 5 | pM | 1.61 | pg | 1.61 | pg-TEQ | 1.61 | ppt |
| 3.22 μL | 1 | pM | 322 | fg | 322 | fg-TEQ | 0.322 | ppt |

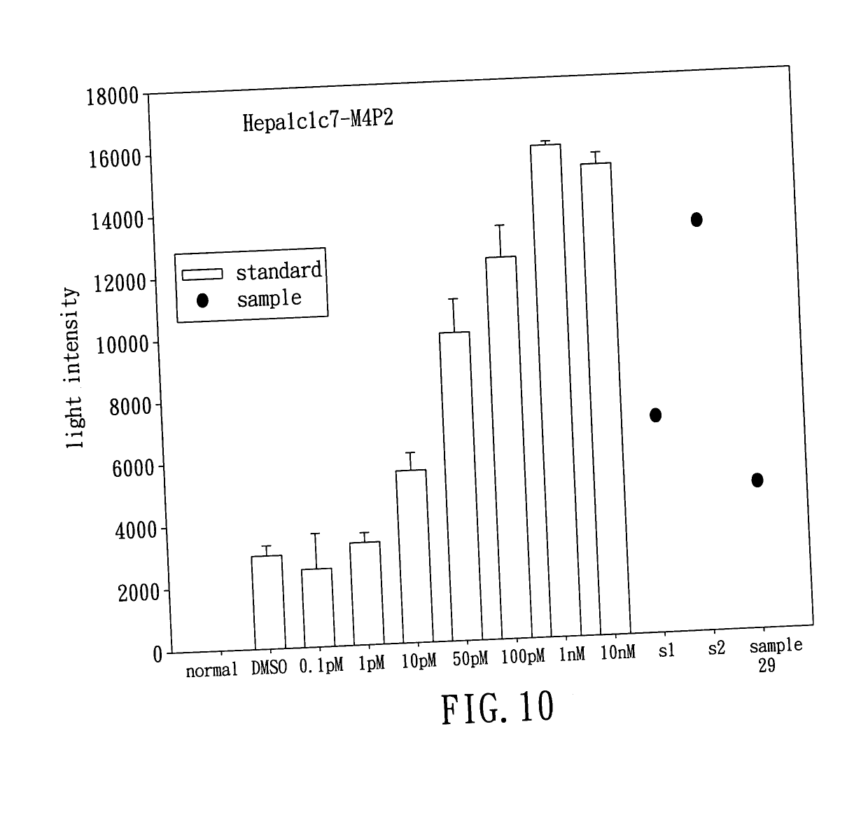

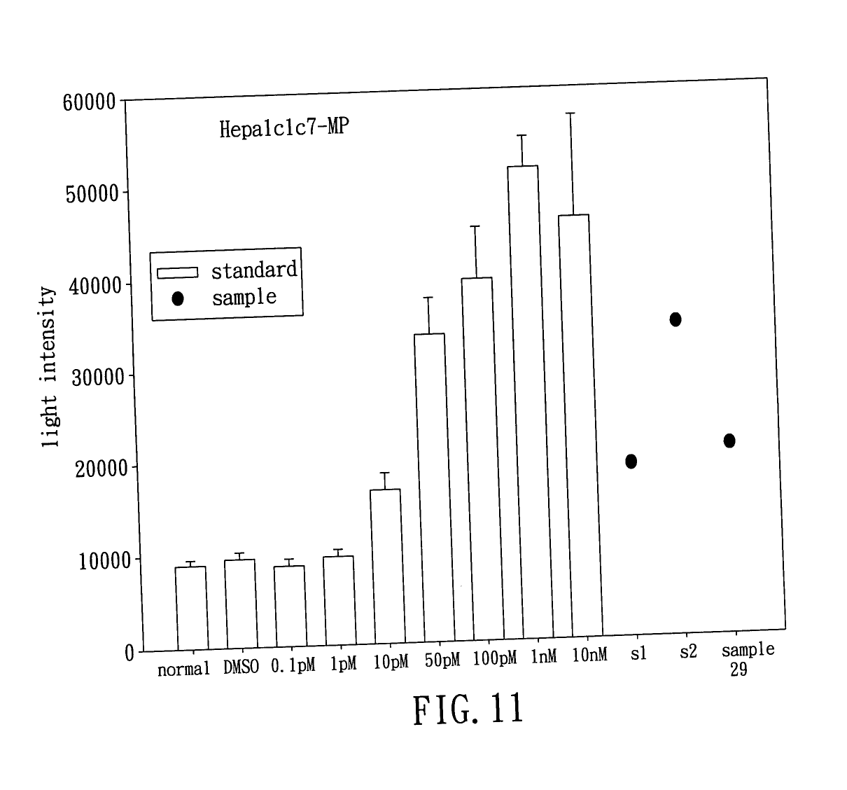

FIGS. 10 and 11 respectively show the light intensity results detected after the transfected mouse hepatoma cell lines Hepa1c1c7-M4P2 and Hepa1c1c7-MP according to this invention were treated with dioxin (2,3,7,8-TCDD) of different concentrations (0.1 pM, 1 pM, 10 pM, 50 pM, 100 pM, 1 nM, and 10 nM), the fly ash sample solution and the two standard samples S₁ and S₂ for 15 hours.

As shown in FIG. 10, when the transfected mouse hepatoma cell line Hepa1c1c7-M4P2 was used to detect dioxin content, a clear positive relationship is observed between dioxin concentration and light intensity. According to FIG. 10, the light intensity of the 40-fold diluted standard sample S₁ is 6980. Thus, judging from the standard dioxin concentrations shown in Table 2, it can be estimated that the mass of dioxin contained in the standard sample S₁ should fall within a range of 3.22˜16.1 pg-2,3,7,8-TCDD. As for the 40-fold diluted standard sample S₂, its light intensity is 13184. Therefore, the mass of dioxin contained therein was reckoned to fall within a range of 32.2-322 pg-2,3,7,8-TCDD. The fluorescence reading of the 40-fold diluted fly ash sample 29 is 4718, so the quantity of dioxin contained therein should fall within a range of 0.322-3.22 pg-2,3,7,8-TCDD.

Since FIG. 10 shows that a clear positive relationship is present between dioxin concentration and light intensity, a linear relationship graph can be plotted using dioxin mass as the abscissa and the detected light intensity as the ordinate. The quantities of dioxin contained in the standard samples S₁ and S₂ and the fly ash sample 29 can be more precisely estimated based on the linear relationship graph to thereby give the following results:
Standard sample S₁: 7.44 pg-2,3,7,8-TCDD;
Standard sample S₂: 104.83 pg-2,3,7,8-TCDD; and
Fly ash sample 29: 2.15 pg-2,3,7,8-TCDD.

Thereafter, by multiplying each of the thus-obtained values by 40, the quantity of dioxin contained in standard sample S₁ was found to be 297.65 pg-2,3,7,8-TCDD, the quantity of dioxin contained in standard sample S₂ was found to be 4193.35 pg-2,3,7,8-TCDD, and the quantity of dioxin contained in the fly ash sample 29 was found to be 85.89 pg-2,3,7,8-TCDD.

Likewise, FIG. 11 shows that when the transfected mouse hepatoma cell line Hepa1c1c7-MP was used to detect dioxin content, a clear positive relationship is observed between dioxin concentration and light intensity. The detected light intensities of the 40-fold diluted two standard samples S₁ and S₂ and the fly ash sample 29 were 18650, 33998 and 20523, respectively. Judging from the dioxin concentration and quantity relationship shown in Table 2, the mass of dioxin contained in the two standard samples S₁ and S₂ and the fly ash sample 29 should respectively fall within the ranges of 3.22-16.1 pg-2,3,7,8-TCDD, 16.1-32.2 pg-2,3,7,8-TCDD, and 3.22-16.1 pg-2,3,7,8-TCDD.

Since FIG. 11 shows that a clear positive relationship is present between dioxin concentration and light intensity, a linear relationship graph can be plotted using dioxin mass as the abscissa and the detected light intensity as the ordinate. The quantities of dioxin contained in the standard samples S₁ and S₂ and the fly ash sample 29 can be more precisely estimated based on the linear relationship graph to thereby give the following results:
Standard sample S₁: 4.81 pg-2,3,7,8-TCDD;
Standard sample S₂: 17.65 pg-2,3,7,8-TCDD; and
Fly ash sample 29: 6.24 pg-2,3,7,8-TCDD.

Thereafter, by multiplying each of the thus-obtained values by 40, the quantity of dioxin contained in standard sample S₁ was found to be 192.36 pg-2,3,7,8-TCDD, the quantity of dioxin contained in standard sample S₂ was found to be 705.97 pg-2,3,7,8-TCDD, and the quantity of dioxin contained in the fly ash sample 29 was found to be 249.536 pg-2,3,7,8-TCDD.

In conclusion, when the transfected mouse hepatoma cell line Hepa1c1c7-M4P2 is used for detection, by referring to the detection results of dioxin (2,3,7,8-TCDD) that serves as a standard, the bioassay result of the standard sample S₁ whose chemical analytical concentration is defined as 0.1 ng-TEQ corresponds to 297.65 pg-2,3,7,8-TCDD, whereas the bioassay result of the standard sample S₂ whose chemical analytical concentration is defined as 0.5 ng-TEQ corresponds to 4193.35 pg-2,3,7,8-TCDD. When the transfected mouse hepatoma cell line Hepa1c1c7-MP is used for detection, the bioassay result of the standard sample S₁ whose chemical analytical concentration is defined as 0.1 ng-TEQ corresponds to 192.36 pg-2,3,7,8-TCDD, whereas the bioassay result of the standard sample S₂ whose chemical analytical concentration is defined as 0.5 ng-TEQ corresponds to 705.97 pg-2,3,7,8-TCDD.

Judging from the above values, it can be seen that the bioassay results are often higher than the chemical analytical results. For the transfected mouse hepatoma cell line Hepa1c1c7-M4P2, the ratio between the bioassay concentration and chemical analytical concentration for 0.1 ng-TEQ of the standard sample S₁ is 3.0, whereas the ratio for 0.5 ng-TEQ of the standard sample S₂ is 8.4. The applicants presumed that during chemical analysis, a loss of the dioxin content occurred in the subsequent purification step(s), to thereby result in a comparatively lower TEQ value. In addition, this may be due to the fact that TEF values of various dioxin compounds obtained with bioassay methods are different from conventional TEF values. Therefore, in the subsequent work, the transfected mouse hepatoma cell line Hepa1c1c7-M4P2 can be used to conduct luciferase activity assay against 17 different dioxin compounds, so as to establish a complete bioassay detection system for detecting dioxin-like compounds.

For the transfected mouse hepatoma cell line Hepa1c1c7-MP, the ratio between the bioassay concentration and chemical analytical concentration for 0.1 ng-TEQ of standard sample S₁ is 1.9, whereas the ratio for 0.5 ng-TEQ of standard sample S₂ is 1.4. Therefore, the transfected mouse hepatoma cell line Hepa1c1c7-MP appears to have a higher detection accuracy than the transfected mouse hepatoma cell line Hepa1c1c7-M4P2.

On the other hand, when the fly ash sample 29 was detected using the transfected mouse hepatoma cell lines Hepa1c1c7-M4P2 and Hepa1c1c7-MP, respectively, the thus-obtained bioassay results respectively corresponded to 85.89 pg-2,3,7,8-TCDD and 249.536 pg-2,3,7,8-TCDD. When the fly ash sample 29 was analyzed using a conventional chemical mass spectrogram, the result obtained was 76 pg-TEQ. If a comparison is made between the bioassay results and the chemical analytical results of the fly ash sample 29, the ratios generated from using the transfected mouse hepatoma cell lines Hepa1c1c7-M4P2 and Hepa1c1c7-MP were 1.1 and 3.3, respectively. Judging from this, bioassay values are often greater than chemical analytical values, which is most possibly due to differences between TEF values obtained with bioassay methods and conventional TEF values. Therefore, there is a need to additionally define a TEF value system suitable for bioassay detection. In addition, the amount of dioxin lost in the chemical analytical steps will also render the TEQ values of the detection results to be relatively low.

Given that the accuracy of detection using the transfected mouse hepatoma cell line Hepa1c1c7-MP was superior to that using the transfected mouse hepatoma cell line Hepa1c1c7-M4P2, in the following examples, the transfected mouse hepatoma cell line Hepa1c1c7-MP was used to analyze biological samples taken from natural environments.

### Example 5

### Detection of Dioxin Content in Fish Meat Sample

3 g of fish meat was taken and subjected to preliminary treatments according to the section of “Preliminary treatments of fat-containing samples,” in which 1.97 g of fat was obtained in the step of “detection of oil content %.” Thus, it was assumed that the oil content % of 3 g of fish meat was 65.7%. The 1.97 g of fat was subsequently subjected to the acid wash step and the purification step using an acid silica gel column described in the section of “Preliminary treatments of fat-containing samples,” and the purification step using a multi-layered silica gel column described in the section “Preliminary treatments of environmental samples” to thereby obtain an eluate. The collected eluate was subjected to nitrogen purge until almost dry, and was finally redissolved in 644 μl of DMSO so as to obtain a fish meat sample solution.

According to the method described in Example 3, 3.22 μl of the fish meat sample solution was taken to treat the cells, and the light intensity changes produced by the treated cells were detected. According to the experimental method described in Example 4 and the dioxin (2,3,7,8-TCDD) concentration and quantity relationship shown in Table 2, the fish meat sample solution was estimated to have a dioxin concentration of 7.5 pM. After conversion, the fish meat sample solution was estimated to have 0.96 pg-2,3,7,8-TCDD.

From the above results, the 3 g of fish meat was estimated to have a dioxin content of 0.32 pg/g-fish meat (or 0.49 pg/g-fat).

In addition, since 2,3,7,8-TCDD was used as the standard in the detection, and since the toxicity equivalency (TEQ) of 2,3,7,8-TCDD is set as 1.00, the dioxin content of the fish meat sample may also be expressed as 0.32 pg-TEQ/g-fish meat (or 0.49 pg-TEQ/g-fat).

### Example 6

### Detection of Dioxin Content in Fish Feed Sample

With reference to the method described in Example 5, 100 g of fish feed was taken to conduct the dioxin content detection. The 100 g of fish feed was detected to contain 7.84 g of fat. Therefore, the oil content % of the fish feed sample was 7.84%. Thereafter, 1 g of the fat was used to carry out the acid wash step and the purification step using an acid silica gel column described in the section of “Preliminary treatments of fat-containing samples” and the purification step using a multi-layered silica gel column described in the section of “Preliminary treatments of environmental samples” to thereby obtain an eluate. The collected eluate was subjected to nitrogen purge until almost dry, and was finally redissolved in 644 μl of DMSO so as to obtain a fish feed sample solution.

According to the method described in Example 3, 3.22 μl of the fish feed sample solution was taken to treat the cells, and the light intensity changes produced by the treated cells were detected. According to the experimental method described in Example 4 and the dioxin (2,3,7,8-TCDD) concentration and quantity relationship shown in Table 2, the fish feed sample solution was estimated to have a dioxin concentration of 25 pM. After conversion, the fish feed sample solution was estimated to have a dioxin concentration of 3.22 pg-2,3,7,8-TCDD.

From the above results, the 100 g of fish feed was estimated to have a dioxin content of 0.25 pg/g-fish feed (or 3.22 pg/g-fat).

In addition, since 2,3,7,8-TCDD was used as the standard in the detection, and since the toxicity equivalency (TEQ) of 2,3,7,8-TCDD is set as 1.00, the dioxin content of the fish feed sample may also be expressed as 0.25 pg-TEQ/g-fish feed (or 3.22 pg-TEQ/g-fat).

All patents and literature references cited in the present specification are hereby incorporated by reference in their entirety. In case of conflict, the present description, including definitions, will prevail.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

1. A primer pair for the cloning of dioxin-responsive DNA fragments, comprising a forward primer and a reverse primer, the forward primer consisting essentially of a nucleotide sequence selected from nucleotide sequences as shown in SEQ ID NO:1 and SEQ ID NO:3, the reverse primer consisting essentially of a nucleotide sequence selected from nucleotide sequences as shown in SEQ ID NO:2 and SEQ ID NO:4.

2. The primer pair as claimed in claim 1, wherein the forward primer consists essentially of a nucleotide sequence as shown in SEQ ID NO:1, and the reverse primer consists essentially of a nucleotide sequence as shown in SEQ ID NO:2.

3. The primer pair as claimed in claim 1, wherein the forward primer consists essentially of a nucleotide sequence as shown in SEQ ID NO:3, and the reverse primer consists essentially of a nucleotide sequence as shown in SEQ ID NO:4.

4. A DNA fragment consisting essentially of a nucleotide sequence which is:
(1) substantially corresponding to the nucleotide sequence of a DNA fragment amplified from polymerase chain reaction using a primer pair as claimed in claim 1 and the nucleotide sequence of a CYP1A1 gene as a template;
(2) substantially corresponding to a nucleotide sequence as shown in SEQ ID NO:5;
(3) substantially corresponding to a nucleotide sequence as shown in SEQ ID NO:6; or
(4) substantially corresponding to a nucleotide sequence which is complementary to the nucleotide sequence of the DNA fragment as defined in (1), or the nucleotide sequence as defined in (2) or (3).

5. The DNA fragment as claimed in claim 4, consisting essentially of a nucleotide sequence which substantially corresponds to the nucleotide sequence as shown in SEQ ID NO:5.

6. The DNA fragment as claimed in claim 4, consisting essentially of a nucleotide sequence which substantially corresponds to the nucleotide sequence as shown in SEQ ID NO:6.

7. A recombinant vector, comprising a reporter gene, and a DNA fragment as claimed in claim 4 which is located upstream of the reporter gene.

8. The recombinant vector as claimed in claim 6, wherein the reporter gene is a luciferase gene.

9. The recombinant vector as claimed in claim 7, wherein the DNA fragment consists essentially of a nucleotide sequence which substantially corresponds to the nucleotide sequence as shown in SEQ ID NO:5.

10. The recombinant vector as claimed in claim 9, which is the recombinant vector M4P2.

11. The recombinant vector as claimed in claim 7, wherein the DNA fragment consists essentially of a nucleotide sequence which substantially corresponds to the nucleotide sequence as shown in SEQ ID NO:6.

12. The recombinant vector as claimed in claim 11, which is the recombinant vector MP.

13. A recombinant cell line produced from the transformation of a host cell with a recombinant vector as claimed in claim 7.

14. The recombinant cell line as claimed in claim 13, wherein the host cell as used in the transformation is mouse hepatoma cell Hepa-1C1-C7.

15. The recombinant cell line as claimed in claim 14, which is Mus musculus (mouse) hepatoma cell Hepa1c1c7-MP deposited in the Biosource Collection and Research Center of Food Industry Research and Development Institute (BCRC of FIRDI) under an accession number BCRC 960207 and in the American Type Culture Collection (ATCC) under an accession number ATCC PTA-6089, or the sub-cultured offspring thereof.

16. The recombinant cell line as claimed in claim 14, which is Mus musculus (mouse) hepatoma cell Hepa1c1c7-M4P2 deposited in the Biosource Collection and Research Center of Food Industry Research and Development Institute (BCRC of FIRDI) under an accession number BCRC 960208 and in the American Type Culture Collection (ATCC) under an accession number ATCC PTA-6090, or the sub-cultured offspring thereof.

17. A bioassay for the detection of dioxin-like compounds present in a sample collected from a natural environment, wherein a recombinant cell line as claimed in claim 13 is used to contact a sample containing dioxin-like compounds, such that the reporter gene carried by the recombinant vector harbored within the recombinant cell line is induced to express a gene product which generates a detectable event, the detectable event acting as an index for qualitative and quantitative analysis of the presence of dioxin-like compounds.

18. The bioassay as claimed in claim 17, wherein the reporter gene carried by the recombinant vector is a luciferase gene, and the detectable event is light intensity produced from the reaction of luciferase and luciferin.

19. The bioassay as claimed in claim 17, wherein the recombinant cell line is Mus musculus (mouse) hepatoma cell Hepa1c1c7-MP deposited in the Biosource Collection and Research Center of Food Industry Research and Development Institute (BCRC of FIRDI) under an accession number BCRC 960207 and in the American Type Culture Collection (ATCC) under an accession number ATCC PTA-6089, or the sub-cultured offspring thereof.

20. The bioassay as claimed in claim 17, wherein the recombinant cell line is Mus musculus (mouse) hepatoma cell Hepa1c1c7-M4P2 deposited in the Biosource Collection and Research Center of Food Industry Research and Development Institute (BCRC of FIRDI) under an accession number BCRC 960208 and in the American Type Culture Collection (ATCC) under an accession number ATCC PTA-6090, or the sub-cultured offspring thereof.
